(19) United States
(12) Patent Application Publication
(10) Pub. No.: US 2015/0090010 A1
(43) Pub. Date: Apr. 2, 2015

(54) METHOD FOR DIAGNOSING HEART FAILURE

(71) Applicant: Chang Gung University, Kwei-Shan Tao-Yuan (TW)

(72) Inventors: Chao-Hung Wang, Keelung (TW); Ming-Shi Shiao, Kwei-Shan Tao-Yuan (TW); Mei-Ling Cheng, Kwei-Shan Tao-Yuan (TW)

(73) Assignee: CHANG GUNG UNIVERSITY, Kwei-Shan Tao-Yuan (TW)

(21) Appl. No.: 14/040,017

(22) Filed: Sep. 27, 2013

(51) Int. Cl. G01N 33/49; G01N 33/493

(52) U.S. Cl. CPC G01N 33/49; G01N 33/493
USPC 73/61.41

(57) ABSTRACT

A method for diagnosing heart failure in a subject is provided. The method includes steps of measuring a biological sample of the subject to obtain an amount of at least one biomarker selected from the group consisting of xanthine, spermidine, propionylcarnitine, butyrylcarnitine and P-cresyl sulfate; and comparing the amount of the at least one biomarker to a reference. Moreover, the present invention relates to a method for staging heart failure or evaluating a prognosis of heart failure in a subject.

### BACKGROUND OF THE INVENTION

1. Field of the Invention

This invention relates to a method for diagnosing heart failure or evaluating a prognosis of heart failure in a subject. Moreover, the present invention also relates to a biomarker or kit for diagnosing heart failure or evaluating a prognosis of heart failure in a subject.

2. Description of Related Art

Heart failure (HF) is a complex clinical syndrome that represents the end stage of various cardiac diseases. In the past few decades, substantial advances have been made in understanding the underlying pathophysiology and hemodynamics, and in the development of novel pharmaceuticals and interventional therapies. Nevertheless, short- and long-term heart failure-related re-hospitalization and mortality remain high, and demand substantial amounts of healthcare resources. The limited effectiveness of current treatment strategy at the late stage of heart failure necessitates novel interventional measures to cult the maladaptive molecular processes at sub-clinical stage and to avoid the progression of heart failure to advanced stages.

A variety of biomarkers for heart failure have been identified. B-type natriuretic peptide (BNP) and the N-terminal fragment of the proprotein have emerged as clinically useful markers for diagnosis and prognosis of heart failure. A recent study showed that natriuretic peptides also provide a prognosis for individuals at moderate risk of cardiovascular disease without overt symptoms. Unfortunately, these biomarkers do not provide additional information on molecular targets for therapeutical interventions. Additionally, application of a single biomarker may not be sufficient for evaluating patients with heart failure, and requires compensations through a combination of multiple molecules.

The etiology of a substantial proportion of heart failure patients remains unexplained according to current knowledge on cardiovascular risk factors. Regardless of the heterogeneous etiologies, the development of heart failure is causally related to the inability of the heart to meet the metabolic demands of the body. The accompanying changes in global metabolism are suggestive of clinical application of heart failure-specific metabolome for diagnostic and prognostic purposes. Current staging on heart failure is based on the consensus developed from American College of Cardiology and the American Heart Association (ACC/AHA), instead of pathogenic mechanism. The ACC/AHA classification of heart failure has four stages. For example, stage A refers to those at risks for heart failure, but who have not yet developed structural heart changes (diabetics, those with coronary disease without prior infarct). Stage B refers to individuals with structural heart disease (i.e. reduced ejection fraction, left ventricular hypertrophy, chamber enlargement), however no symptoms of heart failure have ever developed. Stage C means that patients who have developed clinical heart failure. Stage D is meant to patients with refractory heart failure requiring advanced intervention (biventricular pacemakers, left ventricular assist device, or transplantation).

In addition to heart failure staging by the definition of ACC/AHA, there is another classification to define the functional status of heart failure, called New York Heart Association functional classification (class I to class IV). This classification relates symptoms to everyday activities and the patient's quality of life. Class I: No limitation of physical activity. Ordinary physical activity does not cause undue fatigue, palpitation, or dyspnea (shortness of breath). Class II: Slight limitation of physical activity. Comfortable at rest, but ordinary physical activity results in fatigue, palpitation, or dyspnea. Class III: Marked limitation of physical activity. Comfortable at rest, but less than ordinary activity causes fatigue, palpitation, or dyspnea. Class IV: Unable to carry out any physical activity without discomfort; Symptoms of cardiac insufficiency at rest. If any physical activity is undertaken, discomfort is increased.

Taking advantage of the high throughput and the potential of developing multiple biomarkers, metabolomics is a platform for identification of metabolic signatures associated with pre-heart failure to advanced heart failure subtypes independent of the limitations posed by established traditional risk factors. A thorough understanding of the perturbed metabolism in heart failure, together with advances in nutrigenomic research, potentially moves towards development of personalized preventive measures.

US patent application publication 2012/0286157 A1 disclosed a method for diagnosing heart failure in a subject, wherein the method comprises determining in a sample of the subject the amount of at least one biomarker, such as mannose, hypoxanthine, glutamate, uric acid, aspartate and etc. In addition, it also disclosed the method for identifying whether a subject is in need for a therapy of heart failure or determining whether a heart failure therapy is successful.

Although several biomarkers (such as mannose, hypoxanthine, aspartate and etc.) have been used for diagnosing heart failure, there is still a medical need to find more sensitive and specific biomarkers for diagnosing heart failure especially at the early stage of heart failure and evaluating a prognosis of heart failure.

In the present invention, it was developed to determine the clinical application and significance of metabolomic analysis for diagnosing heart failure and evaluating a prognosis of heart failure, to explore the global sophisticated metabolic perturbation in patients with heart failure, and to provide sensitive evaluation of heart failure at different stages or in regression after therapeutic interventions.

### SUMMARY OF THE INVENTION

In view of the prior art's deficiency, the present invention provides a method for diagnosing heart failure in a subject, comprising steps of: measuring a biological sample of the subject to obtain an amount of at least one biomarker selected from the group consisting of xanthine, spermidine, propionylcarnitine, butyrylcarnitine and P-cresyl sulfate; and comparing the amount of the at least one biomarker to a reference.

In one embodiment of the present invention, the biological sample is selected from the group consisting of blood, plasma, serum and urine.

In one embodiment of the present invention, it further comprises steps of measuring a biological sample of the subject to obtain an amount of amino acid; and comparing the amount of the amino acid to a reference of the amino acid.

In one embodiment of the present invention, the amino acid is selected from the group consisting of glutamine, tyrosine, phenylalanine, histidine, arginine, leucine, tryptophan, threonine, isoleucine, lysine, methionine, valine, and proline.

In one embodiment of the present invention, it further comprises steps of measuring a biological sample of the subject to obtain an amount of hypoxanthine; and comparing the amount of the hypoxanthine to a reference of the hypoxanthine.

In one embodiment of the present invention, it further comprises steps of measuring a biological sample of the subject to obtain an amount of phosphatidylcholine; and comparing the amount of the phosphatidylcholine to a reference of the phosphatidylcholine.

In one embodiment of the present invention, the phosphatidylcholine is selected from the group consisting of phosphatidylcholine diacyl C34:4, phosphatidylcholine acyl-alkyl C36:2, phosphatidylcholine acyl-alkyl C34:2, phosphatidylcholine acyl-alkyl C34:3, phosphatidylcholine diacyl C36:0, phosphatidylcholine diacyl C36:1, phosphatidylcholine diacyl C36:3, phosphatidylcholine diacyl C38:6, phosphatidylcholine diacyl C36:6, phosphatidylcholine diacyl C38:5, phosphatidylcholine diacyl C40:5, phosphatidylcholine diacyl C36:2, phosphatidylcholine acyl-alkyl C36:5, phosphatidylcholine diacyl C38:0, phosphatidylcholine acyl-alkyl C32:3, phosphatidylcholine diacyl C40:4, phosphatidylcholine acyl-alkyl C38:3 and phosphatidylcholine diacyl C42:6.

In one embodiment of the present invention, the phosphatidylcholine is preferably selected from the group consisting of phosphatidylcholine acyl-alkyl C34:2, phosphatidylcholine acyl-alkyl C34:3 and phosphatidylcholine diacyl C34:4.

The present invention further provides a method for staging heart failure in a subject, comprising steps of: measuring a biological sample of the subject to obtain an amount of at least one biomarker selected from the group consisting of xanthine, spermidine and propionylcarnitine; and comparing the amount of the at least one biomarker to a reference.

In one embodiment of the present invention, it further comprises steps of measuring a biological sample of the subject to obtain an amount of amino acid; and comparing the amount of the amino acid to a reference of the amino acid.

In one embodiment of the present invention, the amino acid is selected from the group consisting of glutamine, tyrosine, phenylalanine, histidine, arginine, leucine, tryptophan, threonine, isoleucine, lysine, methionine, valine, and proline.

In one embodiment of the present invention, it further comprises steps of measuring a biological sample of the subject to obtain an amount of hypoxanthine; and comparing the amount of the hypoxanthine to a reference of the hypoxanthine.

In one embodiment of the present invention, it further comprises steps of measuring a biological sample of the subject to obtain an amount of phosphatidylcholine; and comparing the amount of the phosphatidylcholine to a reference of the phosphatidylcholine.

In one embodiment of the present invention, the phosphatidylcholine is selected from the group consisting of phosphatidylcholine diacyl C34:4, phosphatidylcholine acyl-alkyl C36:2, phosphatidylcholine acyl-alkyl C34:2, phosphatidylcholine acyl-alkyl C34:3, phosphatidylcholine diacyl C36:0, phosphatidylcholine diacyl C36:1, phosphatidylcholine diacyl C36:3, phosphatidylcholine diacyl C38:6, phosphatidylcholine diacyl C36:6, phosphatidylcholine diacyl C38:5, phosphatidylcholine diacyl C40:5, phosphatidylcholine diacyl C36:2, phosphatidylcholine acyl-alkyl C36:5, phosphatidylcholine diacyl C38:0, phosphatidylcholine acyl-alkyl C32:3, phosphatidylcholine diacyl C40:4, phosphatidylcholine acyl-alkyl C38:3 and phosphatidylcholine diacyl C42:6.

In one embodiment of the present invention, the phosphatidylcholine is preferably selected from the group consisting of phosphatidylcholine acyl-alkyl C34:2, phosphatidylcholine acyl-alkyl C34:3 and phosphatidylcholine diacyl C34:4.

The present invention further provides a method for evaluating a prognosis of heart failure in a subject, comprising steps of: measuring a biological sample of the subject to obtain an amount of at least one biomarker selected from the group consisting of xanthine, spermidine, butyrylcarnitine and P-cresyl sulfate; and comparing the amount of the at least one biomarker to a reference.

In one embodiment of the present invention, it further comprises steps of measuring a biological sample of the subject to obtain an amount of amino acid; and comparing the amount of the amino acid to a reference of the amino acid.

In one embodiment of the present invention, the amino acid is an essential amino acid.

In one embodiment of the present invention, the essential amino acid is selected from the group consisting of histidine, isoleucine, leucine, lysine, methionine, phenylalanine, threonine, tryptophan and valine.

In one embodiment of the present invention, the essential amino acid is preferably selected from the group consisting of leucine, threonine and tryptophan.

In one embodiment of the present invention, it further comprises a step of measuring in the biological sample to obtain dimethylarginine and a ratio of dimethylarginine/arginine.

In one embodiment of the present invention, it further comprises a step of measuring in the biological sample to obtain symmetric dimethylarginine and a ratio of symmetric dimethylarginine/arginine.

The present invention further provides a kit for diagnosing heart failure comprising of: a detector for detecting a biomarker selected from the group consisting of xanthine, spermidine, propionylcarnitine, butyrylcarnitine, P-cresyl sulfate and a combination thereof.

In some embodiments of the present invention, metabonomics/metabolomics technology may use multivariate statistical techniques to analyze the highly complex data sets generated by high-throughput spectroscopy, such as nuclear magnetic resonance (NMR) spectroscopy and mass spectrometry (MS). In some aspects of the invention, the combined use of different types of spectroscopic platforms, such as gas chromatography mass spectrometry (GC-MS) and liquid chromatography mass spectrometry (LC-MS), can take advantage of complementary analytical outcomes and therefore, provide a broadened metabolic “window” for explaining the biological variations associated with pathophysiological conditions. In certain aspects of the invention, identifying metabolites that account for the differences between the metabolic profiles of people with heart failure and healthy counterparts can reveal important underlying molecular mechanisms of the disease.

In some embodiments of the present invention, profiling methods may include gas chromatography and mass spectrometry. For example, the profiling methods according to an embodiment of the present invention may include gas chromatography-time-of-flight mass spectrometry (GC-TOFMS) and ultra-performance liquid chromatography-quadrupole time-of-flight mass spectrometry (UPLC-QTOFMS). In certain embodiments, more than one profiling method may be used to obtain data about metabolites in a patient sample. In some embodiments, one or more profiling methods may be used together with multivariate statistical techniques to assess a profile of metabolites in a patient sample.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B show the diagnostic values of global metabolomics for heart failure. The plasma samples from patients at different stages (stages A, stage B, and stage C) of heart failure and normal subjects were collected for determination of global metabolite concentrations by LC-MS/MS. (A) OPLS-DA score plots show the considerable separation between the normal controls, and patients with heart failure at stages A and C. To discriminate the normal controls and patients at stage C by using the whole global metabolomics dataset, a global metabolomics-derived parameter was calculated, called t[1](shown on the x-axis). To discriminate the normal controls and patients at stage A by using the whole global metabolomics dataset, another global metabolomics-derived parameter was calculated, called t[0](shown on the y-axis). On the scale of t[1], the cluster area of patients at stage A is similar to the normal controls, however, shifting up on the scale of t[0] compared to the normal controls. (B) Based on the way how these global metabolomics-derived parameters were calculated, the values of t[1] and t[0] were calculated for patients at stage B. The score plots of patients at stage B span the regions among stage A, stage C and the normal control groups.

FIG. 2 shows metabolic pathways implicated in the pathogenesis of heart failure (HF). Disturbances in the urea cycle (a), biopterin cycle (b), MTA cycle (c), methionine cycle (d), ornithine-proline-glutamate (e), polyamine synthesis (f), dopamine synthesis (g), methylation (creatinine and phosphatidylcholine) (h), transsulfuration (taurine) (i), p-cresyl sulfate synthesis (j) and purine metabolism (k) pathways were identified in HF patients. Metabolites significantly increased in HF patients (red); metabolites significantly decreased in HF patients (blue); metabolites unchanged in HF patients (black) and metabolites not assayed (grey).

FIG. 3 shows serial follow-up after acute heart failure. BNP and tPS[1] levels are shown in 32 patients who survived longer than 12 months and improved significantly with New York Heart Association functional class I at the end of 12 months. N, normal controls; M0, M6 and M12, indicate values at pre-discharge, and 6 and 12 months after discharge, respectively. “tPS[1]”: Based on the combination of four metabolites (Histidine, Phenylalanine, Spermidine and Hypoxanthine), a parameter was produced, called tPS[1].

FIG. 4 shows the diagnostic values of BNP and a few targeted metabolites and targeted metabolites combinations. The ROC curves are shown for the diagnosis of stage C heart failure (versus normal controls) by B-type natriuretic peptide (BNP), t[2], and tPS[2]. “t[2]”: a parameter derived from taking all targeted metabolites into account; “tPS[2]”: Based on the combination of 4 metabolites (histidine, phenylalanine, spermidine, and phosphatidylcholine diacyl C34:4), a parameter was produced, called tPS[2].

FIGS. 5A to 5C show the prognostic value of metabolomics. (A) The ROC curves for comparing the prognostic values of BNP, t[2], tPS[2], and tPS[3]. (B and C) The Kaplan-Meier curves of tPS[3] and BNP, respectively, for predicting a composite event of all-cause death and heart failure-related re-hospitalization. “tPS[3]”: Based on the combination of 4 classes of metabolites (dimethylarginine/Arginine ratio, spermidine, butyrylcarnitine, and total amount of essential amino acid), a parameter was produced, called tPS[3].

### DETAILED DESCRIPTION OF THE PREFERRED EMBODIMENTS

The following specific examples are used for illustrating the present invention. A person skilled in the art can easily conceive the other advantages and effects of the present invention. The present invention can also be implemented by different specific cases be enacted or application, the details of the instructions can also be based on different perspectives and applications in various modifications and changes do not depart from the spirit of the creation.

It is further noted that, as used in this specification, the singular forms “a,” “an,” and “the” include plural referents unless expressly and unequivocally limited to one referent. The term “or” is used interchangeably with the term “and/or” unless the context clearly indicates otherwise.

As used herein, the term “subject” or “individual” may be an animal. For example, the subject or individual may be a mammal. Also, the subject or individual may be a human. The subject or individual may be either a male or female. The subject or individual may also be a patient, where a patient is an individual who is under dental or medical care and/or actively seeking medical care for a disorder or disease.

As used herein, the term “healthy” refers to an individual not having heart failure or other related disorders.

As used herein, the term “metabolism” refers to the set of chemical reaction that occur in a living organism to maintain life. Metabolism is usually divided into two categories: catabolism and anabolism. Catabolism is a set of chemical reactions that breaks down organic matter (e.g., to harvest energy in cellular respiration). Anabolism is a set of chemical reactions that use energy to construct components of cells (e.g., protein and nucleic acid synthesis).

As used herein, the term “biomarker” refers to a molecular species which serves as a distinctive biological or biologically derived indicator (such as a biochemical metabolite in the body) of a process, event, or condition (such as aging, disease, or exposure to a toxic substance).

As used herein, the term “metabolite” is an intermediate or product of metabolism. The term metabolite is generally restricted to small molecules. A “primary metabolite” is a metabolite directly involved in normal growth, development, and reproduction (e.g., alcohol). A “secondary metabolite” is a metabolite not directly involved in those processes, but that usually has an important ecological function (e.g., antibiotics, pigments). Some antibiotics use primary metabolites as precursors, such as actinomycin which is created from the primary metabolite, tryptophan. Rather, for the purposes of the present invention, the term metabolite refers to the small molecules (<1000 Dalton) intermediates and products involved in metabolic pathways such as glycolysis, the citric acid (TCA) cycle, amino acid synthesis and fatty acid metabolism, amongst others.

As used herein, the term “metabolomics” or “metabonomics” refers to the systematic study of metabolite profiles generated by biological processes in a biological system under a given set of conditions. “Metabolome” refers to the complete set of small-molecule metabolites (such as metabolic intermediates, hormones and other signaling molecules, and secondary metabolites) to be found within a biological sample (e.g., a biological cell, tissue, organ or organism) that are the end products of cellular processes. Metabolomics is a platform technology to provide top-down, global, and unbiased information. There are two approaches to metabolomics: global metabolic profiling and targeted metabolomics.

As used herein, the term “metabolite profile” or “metabolite biomarker profile” refers to a panel of metabolites that have been determined to have different levels (e.g., increased or decreased) in healthy subjects as compared to unhealthy subjects (e.g., subject having heart failure) or at different disease states (e.g., different stages of disease).

As used herein, the term “heart failure (HF)” refers to a condition in which the function of the heart is impaired, such that the heart is unable to pump blood at an adequate rate or in adequate volume. Heart failure can be systolic, such that a significantly reduced ejection fraction of blood from the heart and, thus, a reduced blood flow. Therefore, systolic heart failure is characterized by a significantly reduced left ventricular ejection fraction (LVEF), preferably, an ejection fraction of less than 50%. Alternatively, heart failure can be diastolic, i.e. a failure of the ventricle to properly relax that is usually accompanied by a stiffer ventricular wall. The diastolic heart failure causes inadequate filling of the ventricle, and thus affects the blood flow. Thus, diastolic dysfunction also results in elevated end-diastolic pressures. Heart failure may, thus, affect the right heart (pulmonary circulation), the left heart (body circulation) or both. Techniques for measuring heart failure are well known in the art and include echocardiography, electrophysiology, angiography, and the determination of peptide biomarkers, such as the B-type Natriuretic Peptide (BNP) or the N-terminal fragment of its propeptide, in the blood. It is understood that heart failure can occur permanently or only under certain stress or exercise conditions. Typical symptoms of heart failure include dyspnea, chest pain, dizziness, confusion, pulmonary and/or peripheral edema. Heart failure can be classified as stages A, B, C and D according to American College of Cardiology and the American Heart Association 2001 guidelines. Stage A: patients at high risk for developing heart failure in the future but no functional or structural heart disorder. Stage B: a structural heart disorder but no symptoms at any stage. Stage C: previous or current symptoms of heart failure in the context of an underlying structural heart problem, but managed with medical treatment. Stage D: patients with refractory heart failure requiring advanced intervention.

As used herein, the term “global metabolites” refers to obtain a global, extensive metabolite profiling which is used for comparison of a large number of analytes in a specific condition, or across several groups of different conditions. It can be achieved by analyzing replicate samples from different treatment conditions (e.g., drug-treated vs. control group) or different pathophysiological states (e.g. diabetic vs. normal group). For this purpose, biospecimens (cells, plasma, urine, saliva, or pathological specimens) are subjected to analysis (by analytical tools such as LC-MS) to generate datasets that are subsequently subjected to univariate or multivariate statistical analysis. Global metabolomics is aimed to identify features that may systemically differentiate a large number of metabolites into groups (classes).

As used herein, the term “targeted metabolites” refers to the identification and quantification of a defined set of structurally known and annotated metabolites and is based on well established biochemical pathways.

Many examples have been used to illustrate the present invention. The examples sited below should not be taken as a limit to the scope of the invention.

### EXAMPLES

### Materials and Methods for Metabolomics Analysis

### 1. Patients and Study Design:

During the period from January, 2005 to December, 2009, patients at heart failure stages B and C were enrolled in this study. From May, 2008 to December, 2009, patients at heart failure stage A and normal controls were enrolled. Patients at stage C were those hospitalized due to acute cardiogenic pulmonary edema, and aged 20-85 years. Patients with systolic and diastolic heart failure were included. Patients at stage B were those with post-acute myocardial infarction regardless of their left ventricular ejection fraction (LVEF), those with any severe structure abnormalities or those with a LVEF of <40%. But, patients at stage B are asymptomatic. Patients at stage A were (1) those who have an angiogram-documented coronary artery disease, a LVEF of >50% and are asymptomatic; or (2) those who have risk factors, but are asymptomatic and have no angiogram-documented coronary disease. Normal controls were people who were aged 20-85 years, and had no significant systemic disease, such as hypertension, diabetes mellitus, or coronary artery disease. They were not on any medications, and had a LVEF of >60%.

Exclusion criteria included (1) the presence of systemic diseases such as hypothyroidism, decompensated liver cirrhosis, and systemic lupus erythematosus; (2) the presence of disorder other than heart failure that might compromise survival within 6 months; (3) patients being bed-ridden for >3 months and/or unable to stand alone; (4) patients with serum creatinine >3 mg/dl; and (5) patients with severe coronary artery disease without complete revascularization therapy. Informed consent was obtained from all patients. The study was designed and carried out in accordance with the principles of the Declaration of Helsinki and with approval from the Ethics Review Board of Chang Gung Memorial Hospital.

### 2. Blood Sampling and Examination

Blood samples were collected before discharge, and at 6 and 12 months after discharge in EDTA-containing tubes. Plasma was analyzed by metabolomic workflow described in the succeeding section. BNP was measured in triplicate with the Triage BNP Test (Biosite, San Diego, Calif.), which was a fluorescence immunoassay for quantitative determination of plasma BNP. Other measurements, including kidney function, hemoglobin, and C-reactive protein, were conducted in the central core laboratory.

### 3. Disease Management Program

The patients at stages C were taken care by an HF team consisting of three cardiologists specializing in HF care, one psychologist, one dietary assistant, and two case managers.

### 4. Follow-Up Program

Follow-up data were prospectively obtained every month from hospital records, personal communication with the patients' physicians, telephone interviews, and patients' regular visits to staff physician outpatient clinics. “Re-hospitalization” was defined as HF-related re-hospitalizations. A committee of 3 cardiologists adjudicated all hospitalizations without knowledge of patients' clinical variables to determine whether the events are related to worsening HF. “All-cause death” was chosen as an endpoint because of the interrelationship of HF with other comorbidities in the patient cohort. The most severe event was considered an endpoint during the follow-up period. Only was the composite event of HF-related re-hospitalization and all-cause death analyzed for the prognostic purpose.

### 5. Plasma Metabolome Analysis

### (1) Plasma Global Metabolites Analysis by LC-TOFMS

To 50-μl plasma, 200 μl acetonitrile (ACN) was added. The mixture was vortexed for 30 s, sonicated for 15 min. and centrifuged at 10,000×g for 25 min. The supernatant was collected into a separate glass tube. The pellets were re-extracted with 200 μl 50% methanol. The aqueous methanolic supernatant and acetonitrile supernatant were pooled and dried in a nitrogen evaporator. The residues were saved and stored at −80° C. For metabolomics analysis, the residues were suspended in 100 μl of 95:5 water/acetonitrile and centrifuged at 14,000×g for 5 min. The clear supernatant was collected for LC-MS analysis.

Liquid chromatographic separation was achieved on a 100 mm×2.1 mm Acquity 1.7-μm C8 column (Waters Corp., Milford, USA) using a ACQUITY™ UPLC system (Waters Corp., Milford, USA). The column was maintained at 45□, and at a flow rate of 0.5 ml/min. Samples were eluted from LC column using a linear gradient: 0-2.5 min: 1-48% B; 2.5-3 min: 48-98% B; 3-4.2 min: 98% B; 4.3-6 min: 1% B for re-equilibration. The mobile phases were 0.1% formic acid (solvent A) in water and 0.1% formic acid in acetonitrile (solvent B).

The eluent was introduced into the TOF MS system (SYNAPT G1 high-definition mass spectrometer, Waters Corp., Milford, USA) and operated in a ESI-positive ion mode. The conditions were as follows: desolvation gas was set to 700 l/h at a temperature of 300° C., cone gas set to 25 l/h, and source temperature set at 80° C. The capillary voltage and cone voltage were set to 3,000 V and 35 V, respectively. The MCP detector voltage was set to 1,650 V. The data acquisition rate was set at 0.1 s with a 0.02 s interscan delay. The data were collected in centroid mode from 20 to 990 m/z. For accurate mass acquisition, a lock-mass of sulfadimethoxine at a concentration of 60 ng/ml and a flow rate of 60 □l/min (an [M+H]⁺ ion at 311.0814 Da in ESI-positive mode).

Raw mass spectrometric data were processed using MassLynx V4.1 and MarkerLynx software (Waters Corp., Milford, USA). The intensity of each mass ion was normalized with respect to the total ion count to generate a data matrix that included the retention time, m/z value, and the normalized peak area. The multivariate data matrix was analyzed by SIMCA-P software (version 13.0, Umetrics AB, Umea, Sweden). OPLS-DA models were carried out prior to the Pareto scaling was applied. SIMCA-P had been used for multivariate data analysis and representation.

Exact molecular mass data which showed significant differences between two groups, were then submitted for database searching, either in-house or using the online HMDB (http://www.hmdb.ca/) and KEGG (http://www.genome.jp/kegg/) databases. For identification of specific metabolites, standards were subject to UPLC-MS/MS analyses under the conditions identical to those of the profiling experiment. MS/MS spectra were collected at 0.1 spectra per second, with a medium isolation window of ˜4 m/z. The collision energy is set from 5 to 35 V.

The construction, interaction, and pathway analysis of potential biomarkers was performed with MetaboAnalyst software based on database source including the KEGG and HMDB, to identify the affected metabolic pathways analysis and visualization. The possible biological roles were evaluated by the enrichment analysis.

### (2) Quantitation of Plasma Targeted Metabolites (Concentrations Determination)

The targeted metabolite analyses were carried out with the AbsoluteIDQ® p180 Kit (Biocrates Life Science AG Innsbruck, Austria). The kit was used to identify and quantify 184 metabolites covering five metabolite classes, namely 90 glycerophospholipids and 15 sphingolipids (76 phosphatidylcholines, 14 lysophosphatidylcholines and 15 sphingomyelines), 19 biogenic amines, 40 acyl carnitines, 19 amino acids, and hexose. Each 10 μL plasma sample was mixed with isotopically labeled internal standards in a 96-well multititer plate, and dried under a stream of nitrogen. Amino acids and biogenic amines were derivatized with 5% phenylisothiocyanate (PITC) for 20 min, and subsequently dried under nitrogen. Three hundred μL of extraction solution (5 mM ammonium acetate in methanol) was added, and after 30 min incubation, the mixture was centrifuged for 2 min at 100×g. Subsequently, a 150-μL aliquot of filtrate was transferred to a microtiter plate, and diluted with 150 μL of water for analysis of amino acids and biogenic amines by LC-MS/MS. The remaining filtrate was mixed with 400 μl of kit MS running solvent for flow injection analysis coupled with tandem mass spectrometric analysis (FIA-MS/MS). The analysis was performed in positive and negative electrospray ionization modes. Identification and quantification were achieved by multiple reaction monitoring (MRM) and standardized by spiking in of isotopically-labeled standards. In LC-MS analysis, the MS was coupled to an UPLC (Waters Corp, Milford, USA), and the metabolites were separated on a reverse phase column (2.1 mm×50 mm, BEH C18, Waters Corp, Milford, USA). The mobile phases were composed of a gradient mixture of solvent A (0.2% formic acid in water) and solvent B (0.2% formic acid in acetonitrile) (0 min 0% B, 3.5 min 60% B, 3.8 min 0% B, 3.9 min 0% B). Elution was performed at a flow rate of 900 μL/min. The column temperature was maintained at 50° C. For FIA, an isocratic method was used, kit MS running solvent as the mobile phase, with varying flow conditions (0 min, 30 μL/min; 1.6 min 30 μL/min; 2.4 min, 200 μL/min; 2.8 min, 200 μL/min; 3 min 30 μL/min). The corresponding MS settings were as follows: dwell time 0.019-0.025 sec; 3.92 KV voltage for positive mode; 1.5 KV for negative mode; nitrogen as collision gas medium; source temperature 150° C. The parameters for LC-MS were: dwell time 0.006-0.128 s; source temperature 150° C.; 3.20 KV voltage; nitrogen as collision gas medium. Data import and pre-processing steps for targeted MS data analyses were done using TargetLynx (Waters, Mass., USA). The integrated MetIDQ software (Biocrates, Innsbruck, Austria) was applied to streamline data analysis by automated calculation of metabolite concentrations.

(3) Plasma p-Cresyl Sulfate and Indoxyl Sulfate Quantification

Plasma samples (10 μL) were prepared by protein precipitation with 500 μL methanol (40 ng/ml d4-indoxyl sulfate as an internal standard) followed by centrifugation at 12,00×g for 10 min at 4° C. The supernatant was collected for p-cresyl sulfate and indoxyl sulfate analysis. LC-MS/MS was carried out on a Xevo TQ MS Acquity UPLC system (Waters Corp., Milford, USA). Separation was achieved on a reversed-phase Acquity UPLC BEH C18 column (1.7□μm, 100 mm×2.1 mm). The column was maintained at 40° C., and at a flow rate of 0.5 ml/min. Samples were eluted from LC column using a linear gradient: 0-0.5 min: 10-20% B; 0.5-3 min: 20-70% B; 3-3.5 min: 70-98% B; 3.5-5 min: 98% B; 5.1-7 min: 10% B for re-equilibration. The mobile phases were water (solvent A) and methanol (solvent B). Mass spectral ionization, fragmentation, and acquisition conditions were optimized on the tandem quadrupole mass spectrometer by using electrospray ionization (ESI) in the negative mode. The conditions were as follows: desolvation gas was set to 1000 l/h at a temperature of 500° C., cone gas set to 30 l/h, and source temperature set at 150° C. The capillary voltage and cone voltage were set to 800 V and 30 V, respectively. The mass spectrometer was operated in the multiple reaction monitoring (MRM) mode with dwell and interscan delay times of 0.2 and 0.1 s, respectively. Data were collected and processed by use of Masslynx software (version 4.0).

### (4) Statistical Analysis

Results are expressed as the mean±SD for continuous variables and as the number (percentage) for categorical variables. Data were compared by two-sample t-tests, ANOVA and Chi-square, when appropriate. Metabolomics analysis was performed with softwares as specified. To maximize identification of differences in metabolic profiles between groups, the orthogonal projection to latent structure discriminant analysis (OPLS-DA) model was applied, and performed using the SIMCA-P (version 13.0, Umetrics AB, Umea, Sweden). The variable importance in the projection (VIP) value of each variable in the model was calculated to indicate its contribution to the classification. A higher VIP value represents a stronger contribution to the discrimination among groups. The VIP values of those variables greater than 1.0 were considered significantly different. The diagnostic values of metabolomics and BNP for HF were presented by the area under the curve (AUC) of the receiver operating characteristic (ROC) curves.

Follow-up data were collected as scheduled or at the last available visit. ROC curves and Kaplan-Meier analysis were used to determine the predictors of the first defined events (death, or HF-related re-hospitalization). For Kaplan-Meier analysis, the cutoff value was set at the mean of each variable to get the data of “Log Rank”. The AUC and the value of Log Rank were used to demonstrate the prognosis of metabolomics and BNP in patients with HF. All statistical analyses were 2-sided and performed using SPSS software (version 15.0, SPSS, Chicago, Ill., USA). A p value of <0.05 was considered significant.

### Example 1

### Global Metabolomics Analysis for Diagnosing and Staging Heart Failure

### 1. Baseline Characteristics

A total of 234 subjects were enrolled in this example. This included 51 normal subjects and 183 patients at stages A (n=43), B (n=67), and C (n=73). The baseline characteristics and laboratory data are shown in Table 1. In most of the variables, a significant trend of changes was noted from normal controls to patients at stage A, B, and C. Compared to the normal controls, patients at stage C had remarkably higher BNP levels, wider QRS complex, but lower total cholesterol, low and high density lipoprotein cholesterols, sodium, hemoglobin, albumin, and estimated glomerular filtration rate. In age, although there were no significant differences among the patient groups, they were older than the normal controls. In addition, the percentage of male was also higher in the patient groups. Coronary artery disease was the major etiology of HF patients.

TABLE 1
Demographic and laboratory data in the normal controls and patients at stages A, B, and C

| | Normal | Stage A | Stage B | Stage C | |
|---|---|---|---|---|---|
| | n = 51 | n = 43 | n = 67 | n = 73 | P value |
| Age (years) | 55.2 ± 4.4 | 60.1 ± 10.8 | 59.9± 12.8 | 64.1 ± 12.8 | <0.01 |
| Male (%) | 19 (37.3) | 35 (81.4) | 57 (85.1) | 41 (56.2) | <0.01 |
| LVEF (%) | 72.3 ± 8.0 | 70.1 ± 8.9 | 50.5 ± 14.1 | 37.2 ± 15.6 | <0.01 |
| Blood pressure (mm Hg) | | | | | |
| Systolic | 125.7 ± 15.7 | 124.3 ± 16.8 | 123.4 ± 19.9 | 124.7 ± 19.7 | 0.926 |
| Diastolic | 75.6 ± 12.1 | 76.7 ± 11.8 | 77.1 ± 12.8 | 72.0 ± 12.5 | 0.072 |
| Heart rate, beats/min | 72.3 ± 11.3 | 76.7 ± 11.3 | 73.6 ± 9.2 | 79.5 ± 14.1 | 0.003 |
| Co-morbidity | | | | | |
| Diabetes mellitus (%) | 0 (0) | 18 (41.9) | 22 (32.8) | 38 (52.1) | <0.01 |
| Chronic kidney disease (%) | 0 (0) | 11 (25.6) | 16 (23.9) | 27 (37) | <0.01 |
| Hypertension (%) | 0 (0) | 31 (72.1) | 48 (71.6) | 54 (74.0) | <0.01 |
| Atrial fibrillation (%) | 0 (0.) | 3 (7.0) | 7 (10.4) | 21 (28.8) | <0.01 |
| COPD (%) | 0 (0) | 3 (7.0) | 2 (3.0) | 12 (16.4) | 0.002 |
| Ischemic (%) | 0 (0) | 31 (72.1) | 59 (8.1) | 44 (60.3) | <0.01 |
| Body mass index (kg/m²) | 24.4 ± 3.3 | 26.1 ± 4.2 | 25.8 ± 4.3 | 24.9 ± 4.6 | 0.136 |
| Medication | | | | | |
| ACEI or ARB (%) | 0 (0) | 15 (34.9) | 48 (71.6) | 62 (84.9) | <0.01 |
| β-Blocker (%) | 0 (0) | 18 (41.9) | 53 (79.1) | 51 (69.9) | <0.01 |
| Digoxin (%) | 0 (0) | 0 (0) | 5 (7.5) | 21 (28.8) | <0.01 |
| Diuretic (%) | 0 (0) | 4 (9.3) | 15 (22.4) | 49 (67.1) | <0.01 |
| Laboratory data | | | | | |
| B-type natriuretic peptide (pg/ml) | 9.2 ± 8.8 | 43.0 ± 69.4 | 209.2 ± 359.7 | 851.6 ± 793.8 | <0.01 |
| Cholesterol (mg/dl) | 214.7 ± 35.5 | 175.1 ± 43.0 | 182.3 ± 41.9 | 169.2 ± 58.6 | <0.01 |
| Triglyceride (mg/dl) | 99.2 ± 55.9 | 186.3 ± 110.3 | 138.4 ± 100.6 | 120.2 ± 68.6 | <0.01 |
| Low density lipoprotein (mg/dl) | 139.2 ± 30.1 | 104.3 ± 37.8 | 112.9 ± 39.8 | 108.1 ± 52.1 | <0.01 |
| High density lipoprotein (mg/dl) | 55.3 ± 12.9 | 39.5 ± 11.3 | 42.2 ± 13.3 | 38.6 ± 17.8 | <0.01 |
| Serum sodium (mEq/L) | 140.4 ± 1.3 | 138.5 ± 3.0 | 139.3 ± 2.2 | 138.6 ± 4.1 | 0.003 |
| Hemoglobin (g/dL) | 13.9 ± 1.2 | 13.9 ± 1.6 | 13.8 ± 1.9 | 12.6 ± 2.1 | <0.01 |
| Total bilirubin (mg/dL) | 0.8 ± 0.3 | 0.8 ± 0.3 | 0.8 ± 0.4 | 1.1 ± 0.6 | <0.01 |
| Albumin (g/dl) | 4.4 ± 0.2 | 4.1 ± 0.3 | 3.9 ± 0.3 | 3.5 ± 0.6 | <0.01 |
| Hemoglobin Alc (%) | 5.7 ± 0.3 | 6.6 ± 1.4 | 6.5 ± 1.4 | 6.7 ± 1.5 | <0.01 |
| eGFR (ml/min/1.73 m²) | 99.1 ± 19.2 | 78.6 ± 24.6 | 79.7 ± 32.2 | 65.6 ± 27.9 | <0.01 |
| QRS complex, msec | 89.6 ± 8.6 | 94.3 ± 15.6 | 96.9 ± 19.4 | 106.3 ± 24.9 | <0.01 |

ACEI, angiotensin-converting enzyme inhibitor; ARB, angiotensin receptor blocker; BMI, body mass index; COPD, chronic obstructive pulmonary disease; Chronic kidney disease, estimated glomerular filtration rate (eGFR) <60 ml/min/1.73 m2; LVEF, left ventricular ejection fraction; MDP, multidisciplinary disease management group.

### 2. Global Metabolites Analysis in Heart Failure and Normal Controls

Global metabolites analysis was performed in this example to separate patients at stages A, B, and C, from the normal controls.

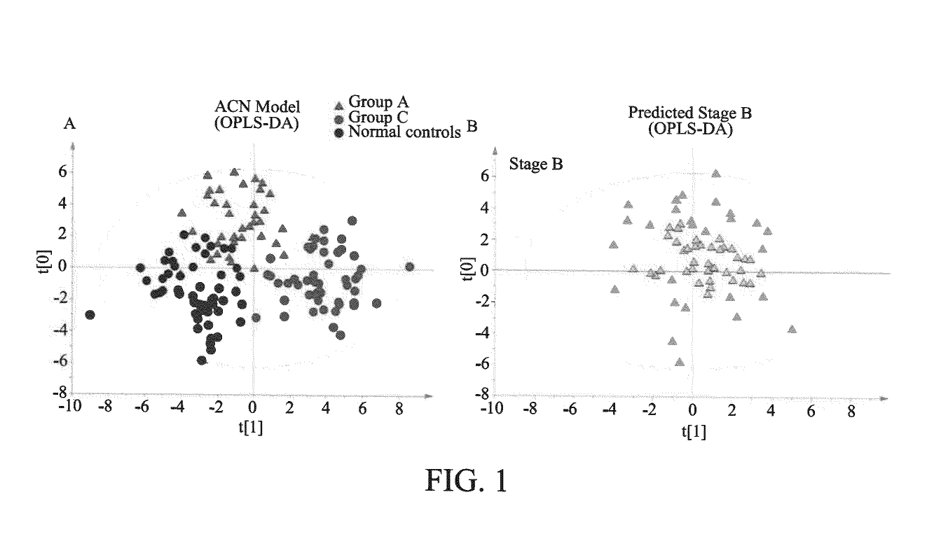

The OPLS-DA remarkably discriminated the normal controls, and patients at stages A and C in the global metabolites analysis (FIG. 1A). In the comparisons among the normal controls, and patients at stages A, B, and C, the metabolites with a VIP score >1.0 are shown in Table 2. To discriminate the normal controls and patients at stage C by using the whole global metabolomics dataset, a global metabolomics-derived parameter was calculated, called t[1](shown on the x-axis). To discriminate the normal controls and patients at stage A by using the whole global metabolomics dataset, another global metabolomics-derived parameter was calculated, called t[0](shown on the y-axis). On the scale of t[1], the score plots cluster area of patients at stage A is similar to the normal controls, however, shifting up on the scale of t[0] compared to the normal controls (FIG. 1A).

Based on the way how these global metabolomics-derived parameters were calculated, the values of t[1] and t[0] were calculated for patients at stage B. The score plots of patients at stage B span the regions among stage A, stage C and the normal control groups (FIG. 1B).

TABLE 2
Statistical analysis of metabolites (with a VIP of >1.0) between patients at stage C (C) and
normal controls (N), and between patients at stage A (A) and normal controls (N), and between
patients at stage B (B) and normal controls (N).

| | | Fold | | Fold | | Fold | |
|---|---|---|---|---|---|---|---|
| | | Change | | Change | | Change | |
| Metabolite ID | M/z | (C/N) | P-value¹ | (A/N) | P-value² | (B/N) | P-value³ |
| Spermine | 203.2235 | 5.9020 | <0.0001 | 1.0588 | <0.05 | 2.0784 | <0.0001 |
| Xanthine | 153.0412 | 4.8854 | <0.0001 | 2.7859 | <0.0001 | 3.7723 | <0.0001 |
| Oleamide | 282.2797 | 4.8534 | 0.0088 | 2.7847 | <0.0001 | 3.7255 | <0.0001 |
| Decatrienoylcarnitine | 310.2016 | 4.4185 | <0.0001 | 2.6943 | <0.0001 | 3.5773 | <0.0001 |
| Hypoxanthine | 137.0463 | 3.2584 | <0.0001 | 4.8133 | <0.0001 | 2.9664 | <0.0001 |
| Glutamate | 148.0610 | 2.7740 | <0.0001 | 3.6739 | <0.0001 | 2.8397 | <0.0001 |
| Pyroglutamic acid | 130.0504 | 2.3127 | <0.0001 | 1.3353 | <0.001 | 2.1514 | <0.0001 |
| P-Cresyl sulfate | 187.0065 | 2.2620 | 0.002 | 1.3581 | 0.004 | 1.7175 | 0.004 |
| Octadecadienylcarnitine | 424.3427 | 1.9810 | <0.0001 | 1.5094 | <0.0001 | 1.2264 | <0.05 |
| Butyrylcarnitine | 232.1549 | 1.9418 | <0.0001 | 1.5241 | <0.0001 | 1.1803 | <0.05 |
| Creatinine | 114.0667 | 1.7820 | <0.0001 | 1.3404 | <0.0001 | 1.5646 | <0.0001 |
| Indoxyl Sulfate | 212.0017 | 1.6930 | 0.008 | 1.1258 | 0.02 | 1.4749 | 0.003 |
| Hydroxybutyrylcarnitine | 248.1498 | 1.6330 | <0.0001 | 1.3333 | <0.05 | 1.2000 | <0.05 |
| Theophylline | 181.0725 | 1.5251 | 0.0126 | 1.3112 | 0.0126 | 1.3722 | <0.01 |
| Butrylcarnitine | 230.1392 | 1.5110 | <0.0001 | 1.2361 | <0.0001 | 1.0667 | <0.05 |
| Octadecenoylcarnitine | 426.3583 | 1.5000 | <0.0001 | 1.2213 | <0.0001 | 1.0246 | <0.05 |
| Ornithine | 133.0977 | 1.4750 | <0.0001 | 0.9511 | <0.05 | 1.1969 | <0.01 |
| Valerycarnitine | 246.1705 | 1.4750 | 0.002 | 1.1287 | 0.002 | 1.4059 | <0.0001 |
| Propionylcarnitine | 218.13868 | 1.4518 | <0.0001 | 1.4778 | <0.0001 | 1.1373 | <0.01 |
| Spermidine | 146.1657 | 1.4420 | <0.0001 | 0.9496 | <0.05 | 1.0426 | <0.05 |
| Phenylalanine | 166.0868 | 1.3400 | <0.0001 | 1.3523 | <0.0001 | 1.1999 | <0.01 |
| Hexose | 179.0555 | 1.3230 | <0.0001 | 1.2690 | <0.0001 | 1.2261 | <0.005 |
| Tetradecenoylcarnitine | 370.2957 | 1.3100 | 0.01 | 1.0238 | <0.05 | 1.0714 | 0.03 |
| Taurine | 126.0225 | 1.3050 | 0.001 | 0.9082 | <0.05 | 1.1026 | 0.01 |
| Proline | 116.07061 | 1.2752 | <0.0001 | 1.3721 | <0.0001 | 1.2105 | <0.0001 |
| Octenoylcarnitine | 286.2018 | 1.2648 | 0.0002 | 1.2558 | 0.0001 | 1.2015 | 0.0002 |
| Prolinebetaine | 144.1024 | 1.2585 | 0.0740 | 1.5277 | <0.005 | 1.2003 | <0.01 |
| Choline | 105.1153 | 1.2265 | <0.0001 | 1.5128 | <0.0001 | 1.1944 | <0.01 |
| Phosphatidylcholine acyl-alkyl C44:5 | 878.7002 | 1.2140 | 0.005 | 1.0607 | <0.05 | 1.0130 | 0.03 |
| Hydroxypropionylcarnitine | 234.1336 | 1.1954 | <0.0001 | 1.4776 | <0.0001 | 1.4286 | <0.0001 |
| Tyrosine | 182.0817 | 1.1800 | 0.001 | 0.9493 | <0.05 | 1.0567 | 0.03 |
| Glycine | 76.0398 | 1.1610 | 0.005 | 0.8737 | 0.002 | 0.9984 | 0.04 |
| Isoleucine | 132.1024 | 1.1610 | 0.006 | 1.2120 | <0.001 | 1.2198 | 0.001 |
| Phosphatidylcholine diacyl C32:0 | 734.5700 | 1.1200 | 0.007 | 0.9955 | <0.05 | 0.9571 | 0.03 |
| Uric acid | 169.0361 | 1.1176 | <0.0001 | 1.3328 | <0.0001 | 1.2132 | <0.0001 |
| Tiglylcarnitine | 244.1543 | 1.1157 | <0.0001 | 1.5772 | <0.0001 | 1.0000 | 0.87 |
| Essential amino acids | — | 1.1052 | <0.0001 | 1.6377 | <0.0001 | 1.0900 | <0.05 |
| Methionine | 150.0583 | 0.9256 | 0.024 | 0.7983 | <0.0001 | 1.0389 | 0.04 |
| Tetradecanoylcarnitine | 372.3114 | 0.9000 | 0.006 | 0.9206 | <0.05 | 0.9048 | 0.01 |
| Phosphatidylcholinediacyl C30:0 | 706.5387 | 0.8940 | 0.017 | 0.9555 | <0.05 | 0.8664 | 0.001 |
| Phosphatidylcholine acyl-alkyl C38:4 | 796.6220 | 0.8890 | 0.018 | 0.9614 | <0.05 | 0.8581 | 0.003 |
| Phosphatidylcholine acyl-alkyl C38:5 | 794.6063 | 0.8880 | 0.02 | 0.9050 | <0.05 | 0.8243 | 0.005 |
| Phosphatidylcholine acyl-alkyl C38:2 | 800.6533 | 0.8740 | 0.02 | 0.8588 | <0.05 | 0.8301 | 0.001 |
| Phosphatidylcholine diacyl C34:2 | 758.5700 | 0.8720 | 0.005 | 0.8914 | 0.003 | 0.8714 | 0.02 |
| Phosphatidylcholine acyl-alkyl C30:1 | 690.5437 | 0.8640 | 0.004 | 0.8701 | 0.001 | 0.8019 | <0.0001 |
| Phosphatidylcholine acyl-alkyl C38:1 | 802.6689 | 0.8590 | 0.005 | 0.8574 | 0.001 | 0.8351 | 0.001 |
| L-Acetylcarnitine | 204.1236 | 0.8578 | <0.0001 | 0.8773 | <0.0001 | 0.9336 | <0.01 |
| Arginine | 175.1195 | 0.8560 | 0.021 | 0.9121 | <0.05 | 1.0013 | 0.1 |
| Phosphatidylcholine diacyl C42:6 | 862.6326 | 0.8530 | 0.002 | 0.9560 | <0.05 | 0.8881 | 0.001 |
| Glutamine | 147.0769 | 0.8510 | 0.001 | 0.8009 | <0.001 | 0.8722 | 0.001 |
| Phosphatidylcholine acyl-alkyl C32:2 | 716.5594 | 0.8500 | 0.015 | 0.8234 | <0.0001 | 0.7136 | <0.0001 |
| Phosphatidylcholine acyl-alkyl C40:6 | 820.6220 | 0.8470 | 0.005 | 0.8139 | <0.0001 | 0.7879 | <0.0001 |
| Phosphatidylcholine acyl-alkyl C30:0 | 692.5594 | 0.8460 | 0.003 | 0.8824 | <0.005 | 0.7978 | <0.0001 |
| Octanoylcarnitine | 288.2175 | 0.8380 | 0.014 | 0.8731 | 0.03 | 0.8731 | 0.001 |
| Phosphatidylcholine diacyl C38:4 | 810.6013 | 0.8350 | 0.003 | 1.0890 | 0.01 | 0.9346 | 0.005 |
| Phosphatidylcholine diacyl C28:1 | 676.4917 | 0.8340 | 0.001 | 0.9123 | 0.01 | 0.8138 | <0.0001 |
| Phosphatidylcholine diacyl C38:6 | 806.5700 | 0.8330 | 0.013 | 0.9321 | 0.02 | 0.8750 | <0.0001 |
| Phosphatidylcholine diacyl C36:3 | 784.5856 | 0.8310 | <0.0001 | 1.0323 | <0.05 | 0.9398 | <0.01 |
| Phosphatidylcholine acyl-alkyl C36:2 | 772.6220 | 0.8270 | <0.0001 | 0.8468 | <0.0001 | 0.7976 | <0.0001 |
| Phosphatidylcholinediacyl C38:0 | 818.6639 | 0.8190 | 0.003 | 0.8103 | <0.0001 | 0.7697 | <0.0001 |
| Phosphatidylcholine acyl-alkyl C36:4 | 768.5907 | 0.8160 | 0.001 | 0.8949 | 0.003 | 0.7891 | <0.0001 |
| Phosphatidylcholine acyl-alkyl C36:3 | 770.6063 | 0.8160 | <0.0001 | 0.8352 | <0.0001 | 0.7576 | <0.0001 |
| Phosphatidylcholine acyl-alkyl C36:5 | 766.5750 | 0.8150 | 0.002 | 0.8637 | 0.003 | 0.7856 | <0.0001 |
| Tryptophan (fragment) | 188.0697 | 0.8139 | <0.0001 | 0.8620 | <0.0001 | 0.8335 | <0.0001 |
| Phosphatidylcholine diacyl C34:3 | 756.5543 | 0.8120 | 0.003 | 0.8950 | 0.002 | 0.8458 | <0.0001 |
| Phosphatidylcholine diacyl C42:5 | 864.6482 | 0.8090 | 0.003 | 0.8723 | 0.005 | 0.8262 | <0.0001 |
| Phosphatidylcholine acyl-alkyl C40:1 | 830.7002 | 0.8070 | 0.001 | 0.8211 | <0.0001 | 0.8218 | <0.0001 |
| Phosphatidylcholine diacyl C40:6 | 834.6013 | 0.8050 | 0.004 | 0.8863 | 0.003 | 0.8621 | <0.0001 |
| Phosphatidylcholine diacyl C40:4 | 838.6326 | 0.8050 | <0.0001 | 1.0210 | <0.05 | 0.8557 | <0.0001 |
| Phosphatidylcholine diacyl C38:3 | 812.6169 | 0.8020 | <0.0001 | 1.0002 | 0.04 | 0.9272 | <0.001 |
| Betaine | 118.0868 | 0.7928 | <0.0001 | 0.7574 | <0.0001 | 0.7279 | <0.0001 |
| Phosphatidylcholine acyl-alkyl C34:2 | 744.5907 | 0.7920 | <0.0001 | 0.7779 | <0.0001 | 0.7253 | <0.0001 |
| Carnitine | 162.1130 | 0.7891 | <0.0001 | 1.1229 | <0.0001 | 1.0993 | 0.04 |
| Phosphatidylcholine diacyl C36:1 | 788.6169 | 0.7890 | <0.0001 | 0.9023 | <0.05 | 0.8522 | <0.0001 |
| Decanoylcarnitine | 316.2488 | 0.7840 | 0.002 | 0.8561 | <0.0001 | 0.8201 | <0.0001 |
| Phosphatidylcholine acyl-alkyl C34:3 | 742.5750 | 0.7840 | 0.001 | 0.7399 | <0.0001 | 0.7068 | <0.0001 |
| Histidine | 156.0773 | 0.7830 | <0.0001 | 0.8303 | <0.0001 | 0.8799 | <0.001 |
| Sphingomyelin C20:2 | 756.1190 | 0.7800 | <0.0001 | 0.9223 | <0.01 | 0.8123 | <0.0001 |
| Phosphatidylcholine acyl-alkyl C38:6 | 792.5907 | 0.7770 | <0.0001 | 0.7949 | <0.0001 | 0.7334 | <0.0001 |
| Phosphatidylcholine diacyl C36:0 | 790.6326 | 0.7740 | <0.0001 | 0.8538 | <0.0001 | 0.8029 | <0.0001 |
| LysoPhosphatidylcholine acyl C 16:0 | 496.3403 | 0.7687 | 0.0002 | 1.0143 | 0.02 | 1.0327 | 0.03 |
| Phosphatidylcholine diacyl C36:2 | 786.6013 | 0.7640 | <0.0001 | 0.8470 | <0.0001 | 0.8136 | <0.0001 |
| Phosphatidylcholine diacyl C38:5 | 808.5856 | 0.7610 | <0.0001 | 0.9651 | <0.05 | 0.8378 | <0.0001 |
| Phosphatidylcholine diacyl C40:5 | 836.6169 | 0.7480 | <0.0001 | 0.9421 | <0.05 | 0.8316 | <0.0001 |
| Phosphatidylcholine diacyl C33:3 | 742.5387 | 0.7450 | <0.0001 | 0.7965 | <0.0001 | 0.7532 | <0.0001 |
| Pipecolic acid | 130.0868 | 0.7437 | <0.0001 | 0.7805 | <0.0001 | 0.7587 | <0.0001 |
| Phosphatidylcholine acyl-alkyl C38:0 | 804.6846 | 0.7280 | 0.008 | 0.7808 | <0.0001 | 0.7692 | <0.0001 |
| Phosphatidylcholinediacyl C36:5 | 780.5543 | 0.6740 | 0.001 | 0.8210 | <0.0001 | 0.7337 | <0.0001 |
| LysoPhosphatidylcholine acyl C18:0 | 524.3716 | 0.6494 | <0.0001 | 0.8996 | <0.001 | 0.9093 | <0.001 |
| LysoPhosphatidylcholine acyl C18:2 | 520.3403 | 0.6435 | <0.0001 | 0.9957 | <0.01 | 1.0422 | 0.04 |
| Caffeine | 195.0882 | 0.6418 | 0.0488 | 0.6822 | 0.02 | 0.7174 | <0.0001 |
| Phosphatidylcholine diacyl C36:6 | 778.5387 | 0.6190 | <0.0001 | 0.7841 | <0.0001 | 0.7203 | <0.0001 |
| Phosphatidylcholine diacyl C34:4 | 754.5387 | 0.6130 | <0.0001 | 0.9723 | <0.01 | 0.7379 | <0.0001 |
| Phosphatidylcholine acyl-alkyl C38:5 | 794.6063 | 0.8880 | 0.02 | 0.9050 | <0.05 | 0.8243 | 0.005 |
| Phosphatidylcholine diacyl C36:6 | 778.5387 | 0.6190 | <0.0001 | 0.7841 | <0.0001 | 0.7203 | <0.0001 |
| Phosphatidylcholin diacyl C34:4 | 754.5387 | 0.6130 | <0.0001 | 0.9723 | <0.01 | 0.7379 | <0.0001 |
| M_008 | 72.0815 | 0.8319 | <0.0001 | — | — | — | — |
| M_014 | 82.0149 | 0.7686 | <0.0001 | — | — | — | — |
| M_018 | 84.0818 | 0.7004 | 0.0007 | — | — | — | — |
| M_049 | 104.1077 | 1.2265 | 0.0004 | — | — | — | — |
| M_051 | 105.0338 | 1.1593 | <0.05 | — | — | — | — |
| M_052 | 105.0708 | 0.8073 | <0.0001 | — | — | — | — |
| M_055 | 110.0101 | 0.7677 | <0.0001 | — | — | — | — |
| M_090 | 126.0927 | 2.2898 | <0.0001 | — | — | — | — |
| M_095 | 130.0507 | 2.6811 | 0.0032 | — | — | — | — |
| M_100 | 130.1597 | 0.4299 | <0.0001 | — | — | — | — |
| M_102 | 131.0507 | 1.1913 | <0.0001 | — | — | — | — |
| M_110 | 134.0977 | 0.8814 | 0.0234 | — | — | — | — |
| M_113 | 136.0163 | 6.7139 | 0.0398 | — | — | — | — |
| M_129 | 143.9976 | 1.1593 | <0.05 | — | — | — | — |
| M_147 | 149.024 | 0.8658 | <0.0001 | — | — | — | — |
| M_157 | 152.0714 | 22.8339 | 0.0163 | — | — | — | — |
| M_168 | 158.156 | 0.8417 | <0.0001 | — | — | — | — |
| M_207 | 174.0538 | 2.7941 | <0.0001 | — | — | — | — |
| M_214 | 176.1189 | 0.8740 | <0.0001 | — | — | — | — |
| M_227 | 182.9644 | 0.6641 | <0.0001 | — | — | — | — |
| M_235 | 188.0697 | 0.8139 | <0.0001 | — | — | — | — |
| M_239 | 191.021 | 1.2344 | 0.0052 | — | — | — | — |
| M_240 | 191.0416 | 0.7507 | <0.0001 | — | — | — | — |
| M_269 | 205.0298 | 4.8647 | <0.0001 | — | — | — | — |
| M_274 | 207.0163 | 0.7653 | <0.0001 | — | — | — | — |
| M_282 | 209.1192 | 0.9732 | <0.05 | — | — | — | — |
| M_315 | 229.1551 | 3.2227 | 0.0002 | — | — | — | — |
| M_382 | 262.1299 | 0.6037 | 0.0005 | — | — | — | — |
| M_390 | 265.1193 | 2.9553 | 0.0044 | — | — | — | — |
| M_457 | 301.1416 | 0.8767 | <0.0001 | — | — | — | — |
| M_489 | 316.2487 | 0.5370 | <0.0001 | — | — | — | — |
| M_526 | 332.294 | 0.4792 | <0.0001 | — | — | — | — |
| M_535 | 337.0646 | 2.5385 | <0.0001 | — | — | — | — |
| M_570 | 354.1947 | 0.7930 | <0.0001 | — | — | — | — |
| M_571 | 355.0126 | 0.5224 | 0.0001 | — | — | — | — |
| M_610 | 375.0115 | 1.1735 | 0.0238 | — | — | — | — |
| M_618 | 381.1297 | 131.8932 | <0.05 | — | — | — | — |
| M_636 | 390.9849 | 1.1508 | 0.0164 | — | — | — | — |
| M_653 | 402.1797 | 430.4213 | <0.05 | — | — | — | — |
| M_664 | 409.1625 | 0.8294 | <0.0001 | — | — | — | — |
| M_685 | 425.1369 | 0.8488 | <0.0001 | — | — | — | — |
| M_686 | 426.213 | 1.7022 | <0.05 | — | — | — | — |
| M_760 | 520.3401 | 0.6435 | 0.0017 | — | — | — | — |
| M_765 | 526.2943 | 0.1465 | <0.0001 | — | — | — | — |
| M_0031 | 171.1492 | 0.9832 | <0.05 | 0.9967 | 0.8737 | 0.9980 | 0.9257 |
| M_0375 | 104.1072 | 1.4218 | <0.0001 | 1.1121 | 0.0578 | 1.0181 | 0.7367 |
| M_0526 | 260.9942 | 1.4266 | <0.0001 | 1.6292 | 0.0002 | 1.5814 | <0.0001 |
| M_0708 | 185.9643 | 0.8677 | 0.0001 | 0.9628 | 0.1338 | 1.0048 | 0.8473 |
| M_0761 | 189.0869 | 0.7760 | 0.0192 | 0.8589 | 0.0321 | 1.1955 | 0.0583 |
| M_0943 | 209.0555 | 1.5244 | <0.0001 | 1.1174 | 0.0615 | 1.2614 | 0.0207 |
| M_1034 | 169.1075 | 2.9547 | <0.0001 | 1.8887 | 0.0110 | 1.5405 | 0.0315 |
| M_1131 | 130.0863 | 0.7631 | <0.0001 | 0.8427 | 0.0188 | 0.8580 | 0.0252 |
| M_1133 | 84.0809 | 0.6868 | <0.0001 | 0.8004 | 0.0099 | 0.8035 | 0.0117 |
| M_1174 | 147.0443 | 1.1629 | 0.0179 | 1.0552 | 0.2445 | 0.9313 | 0.0835 |
| M_1185 | 248.1491 | 2.5233 | 0.0007 | 1.3870 | 0.0788 | 1.0831 | 0.6350 |
| M_1253 | 136.0153 | 8.7313 | <0.05 | 1.1071 | 0.1616 | 0.9095 | 0.0826 |
| M_1257 | 263.1137 | 8.1430 | <0.05 | 1.0430 | 0.7413 | 1.3713 | 0.0497 |
| M_1326 | 153.0659 | 1.6787 | 0.0406 | 1.2339 | 0.3834 | 1.4802 | 0.0963 |
| M_1463 | 150.0588 | 2.5757 | <0.0001 | 1.4064 | 0.0040 | 1.4420 | 0.0017 |
| M_1465 | 153.0659 | 1.6336 | 0.0114 | 1.1852 | 0.3109 | 1.1744 | 0.2405 |
| M_1611 | 319.1363 | 72.6607 | <0.05 | 1.7386 | 0.3694 | 0.8971 | 0.8219 |
| M_1707 | 146.0601 | 1.3923 | 0.0002 | 0.9956 | 0.9566 | 0.9595 | 0.5402 |
| M_1728 | 120.0814 | 1.2416 | <0.0001 | 1.1452 | 0.0061 | 1.0329 | 0.3064 |
| M_1731 | 93.0701 | 1.2707 | <0.0001 | 1.1065 | 0.0366 | 1.0118 | 0.7153 |
| M_1732 | 77.039 | 1.2538 | <0.0001 | 1.1568 | 0.0036 | 1.0339 | 0.3102 |
| M_1733 | 149.06 | 1.2139 | <0.0001 | 1.1148 | 0.0077 | 1.0326 | 0.2130 |
| M_1871 | 126.0914 | 2.2405 | <0.0001 | 1.3054 | 0.0352 | 1.2222 | 0.1179 |
| M_2020 | 574.9177 | 5.2426 | 0.001 | 3.1454 | 0.2943 | 0.0000 | 1.0000 |
| M_2217 | 167.0335 | 4.9645 | 0.0166 | 3.4615 | 0.1669 | 1.8851 | 0.1975 |
| M_2253 | 236.1834 | 111.2498 | <0.05 | 316.5225 | 0.0411 | 689.2129 | 0.0898 |
| M_2262 | 130.0498 | 1.6234 | 0.0236 | 1.2367 | 0.2023 | 1.1722 | 0.3532 |
| M_2319 | 373.122 | 8176.337 | <0.05 | 6706.942 | 0.3255 | 6784.352 | 0.3211 |
| M_2698 | 357.1271 | 18.5978 | <0.05 | 18.5726 | 0.3249 | 18.7389 | 0.3052 |
| M_2833 | 93.5765 | 0.3091 | 0.0030 | 0.5402 | 0.0883 | 0.7212 | 0.3630 |
| M_3029 | 368.0871 | 31.4366 | <0.05 | 2.2780 | 0.2328 | 1.6483 | 0.1478 |
| M_3184 | 336.0631 | 10.1884 | <0.05 | 1.5120 | 0.3697 | 9.6124 | 0.0097 |
| M_3293 | 314.2325 | 0.6385 | 0.0033 | 0.4211 | <0.0001 | 0.4280 | 0.0000 |
| M_3342 | 389.1235 | 6.3609 | <0.05 | 1.4481 | 0.3614 | 5.0206 | 0.2646 |
| M_3369 | 316.2484 | 0.4181 | <0.0001 | 0.3851 | <0.0001 | 0.3963 | 0.0000 |
| M_3371 | 328.2479 | 0.6430 | 0.0369 | 0.5125 | 0.0014 | 0.5434 | 0.0050 |
| M_3610 | 288.2893 | 29.0521 | 0.0001 | 9.4909 | 0.0046 | 0.8718 | 0.3705 |
| M_3727 | 91.0547 | 5.5180 | <0.05 | 0.9161 | 0.5246 | 1.3875 | 0.4406 |
| M_3728 | 404.2072 | 139.3227 | <0.05 | 1.2042 | 0.6347 | 1.1604 | 0.5660 |
| M_3769 | 356.2791 | 0.5046 | 0.0108 | 0.3819 | 0.0006 | 0.5858 | 0.0465 |
| M_3809 | 358.2947 | 0.6305 | <0.05 | 0.4494 | 0.0154 | 0.7409 | 0.3529 |
| M_3871 | 185.0959 | 104.0675 | <0.05 | 1.1257 | 0.2847 | 1.1538 | 0.2132 |
| M_3872 | 231.1024 | 16.9689 | <0.05 | 0.8612 | 0.0904 | 0.9964 | 0.9743 |
| M_3911 | 316.3205 | 20.3052 | 0.0031 | 5.6069 | 0.0393 | 0.4379 | 0.3228 |
| M_4074 | 209.1171 | 0.6845 | 0.0499 | 0.6871 | 0.0200 | 0.9313 | 0.6958 |
| M_4133 | 268.2633 | 0.9246 | <0.05 | 0.9359 | 0.2256 | 0.9984 | 0.9759 |
| M_4148 | 376.2587 | 0.0020 | <0.0001 | 0.2143 | <0.0001 | 0.6099 | 0.0124 |
| M_4151 | 421.3162 | 0.0040 | <0.0001 | 0.2213 | <0.0001 | 0.6212 | 0.0716 |
| M_4231 | 119.0854 | 1.8895 | 0.0489 | 1.4290 | 0.0186 | 1.0436 | 0.6283 |
| M_4264 | 512.3343 | 1.3509 | 0.0070 | 1.1561 | 0.2250 | 1.1989 | 0.1077 |
| M_4604 | 249.1847 | 0.9847 | <0.05 | 1.0110 | 0.3092 | 1.0066 | 0.6168 |
| M_4780 | 399.2507 | 0.9782 | <0.05 | 0.9902 | 0.5343 | 1.0076 | 0.6723 |
| M_4781 | 299.1614 | 0.9735 | <0.05 | 0.9836 | 0.2881 | 1.0066 | 0.6868 |
| M_4889 | 646.2779 | 0.6875 | 0.0194 | 0.8420 | 0.1843 | 0.8011 | 0.0389 |
| M_4900 | 626.296 | 0.6965 | 0.0035 | 0.8115 | 0.0311 | 0.7268 | 0.0002 |
| M_5003 | 380.2488 | 1.5854 | 0.0004 | 1.0729 | 0.6958 | 1.7496 | <0.0001 |
| M_0116 | 331.8446 | — | — | 1.0948 | <0.0001 | — | — |
| M_0396 | 162.1129 | — | — | 1.0443 | <0.05 | — | — |
| M_0417 | 122.027 | — | — | 2.0851 | <0.0001 | — | — |
| M_0527 | 242.9827 | — | — | 1.2182 | <0.0001 | — | — |
| M_0528 | 301.0738 | — | — | 0.8475 | 0.0008 | — | — |
| M_0667 | 383.1133 | — | — | 2.0964 | <0.0001 | — | — |
| M_0899 | 452.9094 | — | — | 0.8593 | 0.0017 | — | — |
| M_1106 | 146.0271 | — | — | 1.4223 | 0.0008 | — | — |
| M_1176 | 91.0545 | — | — | 0.9009 | 0.0053 | — | — |
| M_1178 | 205.0286 | — | — | 0.7732 | 0.0016 | — | — |
| M_1496 | 86.0968 | — | — | 1.0969 | 0.0436 | — | — |
| M_1639 | 367.1497 | — | — | 3.4365 | 0.0004 | — | — |
| M_1916 | 130.0652 | — | — | 0.8769 | 0.0001 | — | — |
| M_2356 | 340.031 | — | — | 3.1049 | <0.0001 | — | — |
| M_2402 | 260.1854 | — | — | 0.6278 | 0.0047 | — | — |
| M_2435 | 304.2116 | — | — | 0.5018 | <0.0001 | — | — |
| M_2498 | 340.0362 | — | — | 3.1534 | <0.0001 | — | — |
| M_2563 | 121.0281 | — | — | 3.3815 | <0.0001 | — | — |
| M_2803 | 308.0509 | — | — | 666.3764 | <0.0001 | — | — |
| M_2804 | 310.0481 | — | — | 344.3028 | <0.0001 | — | — |
| M_2805 | 198.0316 | — | — | 26.2854 | <0.0001 | — | — |
| M_2806 | 262.0455 | — | — | 11.1538 | 0.0002 | — | — |
| M_3102 | 332.2427 | — | — | 0.3681 | <0.0001 | — | — |
| M_3201 | 235.9849 | — | — | 5.2144 | <0.0001 | — | — |
| M_3205 | 237.9557 | — | — | 18.6739 | <0.0001 | — | — |
| M_3443 | 340.2479 | — | — | 0.3948 | <0.0001 | — | — |
| M_3507 | 342.2635 | — | — | 0.3958 | <0.0001 | — | — |
| M_3759 | 368.2787 | — | — | 0.3769 | <0.0001 | — | — |
| M_3882 | 370.0874 | — | — | 924.6001 | <0.0001 | — | — |
| M_3883 | 372.0844 | — | — | 234.7603 | <0.0001 | — | — |
| M_4313 | 321.1415 | — | — | 4.9596 | <0.0001 | — | — |
| M_4556 | 546.3553 | — | — | 1.1106 | <0.05 | — | — |
| M_4886 | 240.0995 | — | — | 0.8120 | 0.0033 | — | — |
| M_4948 | 502.3707 | — | — | 1.4365 | <0.0001 | — | — |
| M_5000 | 337.1676 | — | — | 1.7275 | <0.0001 | — | — |

Essential AA: essential amino acids.
P-value¹, the difference between stage C and normal controls;
P-value², the difference between stage A and normal controls;
P-value³, the difference between stage B and normal controls.

### 3. Identification of Abnormal Metabolic Pathways in HF

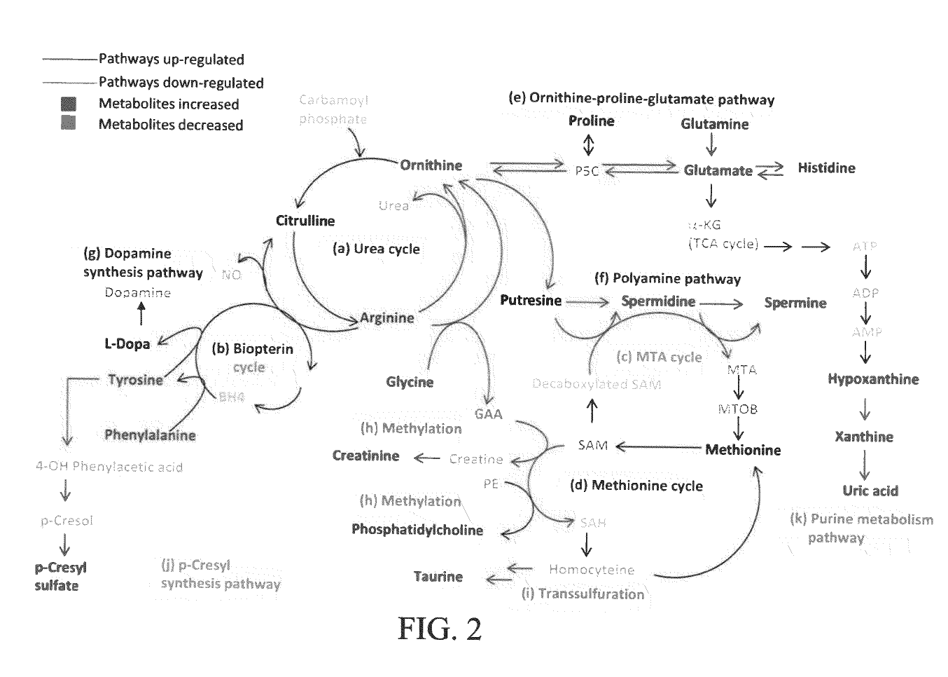

Different categories of metabolites changed at different stages of HF (Table 2). These metabolites include purines, amino acids, biogenic amines, and phospholipids. Compared to the controls, arginine metabolism, urea cycle, purine metabolism, and nitric oxide synthesis pathways were markedly affected in the stage C patients. Levels of some metabolites related to the arginine metabolism, such as glutamine and citrulline, were lowered in the stage C patients. Levels of hypoxanthine, xanthine, uric acid, glutamate, proline, ornithine, spermine and spermidine were elevated in the stage C patients. Aromatic amino acids, such as tyrosine and phenylalanine, were higher in the stage C patients. In addition, levels of several phosphatidylcholines decreased whilst taurine increased. These findings of global metabolites changes in patients at stage C were mapped onto the biochemical pathways through KEGG and HMDB databases (FIG. 2). These findings could demonstrate the abnormalities of metabolites changes behind the clinical presentation of HF, and provide more information related to the disease mechanisms.

### 4. Different Combinations of Global Metabolites to Discriminate Patients at Different Stages of HF, and the Normal Controls

To discriminate patients at stage A from the normal subjects, a few good metabolite combinations were found as shown in the Table 3. The diagnosis values of these combinations are analyzed by receiver operating characteristic (ROC) curves, and are presented by the area under the curve (AUC). The diagnostic values of metabolomics-derived parameters are better than BNP.

TABLE 3
Different combinations of global metabolites to discriminate
patients at stage A from the normal subjects

| | Area under |
|---|---|
| | the curve |
| Metabolites and BNP | (AUC) |
| BNP | 0.762 |
| Gln + PCaeC34:2 + C18:2 + Ile + Hypoxanthine | 0.973 |
| Gln + PCaeC34:2 + C18:2 + Hypoxanthine | 0.971 |
| Gln + PCaeC34:2 + Hypoxanthine | 0.969 |
| Gln + PCaeC34:2 + C3 + TyrPhe + ILe + Hypoxanthine | 0.974 |
| Gln + PCaeC34:2 + C3 + TyrPhe + Hypoxanthine | 0.972 |
| Gln + PCaeC34:2 + C3 + Hypoxanthine | 0.970 |
| Gln + Spermidine + PCaeC34:3 + Hypoxanthine | 0.973 |
| Gln + Spermidine + PCaeC34:3 + Xanthine | 0.974 |
| Gln + Spermidine + PCaeC34:2 + Hypoxanthine | 0.973 |
| Gln + Spermidine + PCaeC34:2 + Xanthine | 0.974 |
| Creatinine + Met + PCaeC34:3 + Essential | 0.972 |
| AA + C5:1 + C3OH + His + Pro | |
| Creatinine + Met + PCaeC34:3 + C5:1 + Pro | 0.933 |
| Creatinine + Met + PCaeC34:3 + Pro | 0.914 |
| Met + PCaeC34:3 + Pro | 0.826 |
| Gln + PCaeC34:2 + C3 + Creatinine | 0.899 |
| Gln + PCaeC34:2 + Creatinine | 0.868 |
| Gln + PCaeC34:2 + C18:2 + Ile | 0.892 |
| Gln + PCaeC34:2 + C18:2 | 0.862 |
| Gln + PCaeC34:2 | 0.806 |
| Gln + PCaeC34:2 + C3 + TyrPhe + Ile | 0.801 |
| Gln + PCaeC34:2 + C3 + TyrPhe | 0.895 |
| Gln + PCaeC34:2 + C3 | 0.869 |

BNP: B-type natriuretic peptide, Met: Methionine; Met: Methionine; Essential AA: Essential amino acid; PCaeC34:3: Phosphatidylcholine acyl-alkyl C34:3, C5:1: Tiglylcarnitine, C3OH: Hydroxypropionylcarnitine, His: Histidine, Pro: Proline, Gln: Glutamine, PCaeC34:2: Phosphatidylcholine acyl-alkyl C34:2, C3: Propionylcarnitine, Tyr: Tyrosine, Phe: Phenylalanine, Ile: Isoleucine, C18:2: Octadecadienylcarnitine.

To discriminate patients at stage A from patients at stage C, a few good metabolite combinations were found as shown in the Table 4. The diagnosis values of these combinations are analyzed by ROC curves, and are presented by the area under the curve (AUC). The diagnostic values of metabolomics-derived parameters are better than BNP.

TABLE 4
Different combinations of global metabolites to discriminate
patients at stage A from patients at stage C

| | Area under | P value |
|---|---|---|
| | the curve | (compared |
| Metabolites and BNP | (AUC) | to BNP) |
| BNP | 0.942 | — |
| Fisher Ratio + His + Phe + Spermidine | 0.966 | 0.05 |
| His + Phe + Spermidine | 0.957 | 0.12 |
| His + Phe + Spermidine + PCaeC34:3 | 0.971 | 0.04 |
| Fisher Ratio + His + Phe + Spermidine + | 0.973 | 0.04 |
| Hypoxanthine | | |
| His + Phe + Spermidine + Hypoxanthine | 0.970 | 0.04 |
| His + Phe + Spermidine + PCaeC34:3 + | 0.972 | 0.04 |
| Hypoxanthine | | |

BNP: B-type natriuretic peptide, Fisher Ratio: ratio of branched chain amino acids to aromatic amino acids, PCaeC34:3: Phosphatidylcholine acyl-alkyl C34:3, His: Histidine, Phe: Phenylalanine.

To discriminate patients at stage C from the normal subjects, a few good metabolite combinations were found as shown in the Table 5. The diagnosis values of these combinations are analyzed by ROC curves, and are presented by the area under the curve (AUC). The diagnostic values of metabolomics-derived parameters are similar to BNP.

TABLE 5
Different combinations of global metabolites to discriminate
patients at stage C from the normal subjects

| | | Area under |
|---|---|---|
| | | the curve |
| Metabolites and BNP | | (AUC) |
| 1. | BNP | 0.971 |
| 2. | His + Phe | 0.815 |
| 3. | His + Phe + Spermidine | 0.969 |
| 4. | His + Phe + Spermidine + PCaaC34:4 | 0.971 |
| 5. | His + Phe + PCaaC34:4 + Hypoxanthine | 0.970 |
| 6. | His + Phe + Hypoxanthine | 0.958 |
| 7. | His + Phe + Spermidine + Hypoxanthine | 0.971 |
| 8. | His + Phe + Spermidine + PCaaC34:4 + Hypoxanthine | 0.973 |

BNP: B-type natriuretic peptide, PCaaC34:4: Phosphatidylcholine diacyl C34:4, His: Histidine, Phe: Phenylalanine.

To discriminate patients at stage B from the normal subjects, a few good metabolite combinations were found as shown in the Table 6. The diagnosis values of these combinations are analyzed by ROC curves, and are presented by the area under the curve (AUC). The diagnostic values of metabolomics-derived parameters are better than BNP.

TABLE 6
Different combinations of metabolites to discriminate
patients at stage B from the normal subjects.

| | Area under | P value |
|---|---|---|
| | the curve | (compared |
| Metabolites | (AUC) | to BNP) |
| BNP | 0.943 | — |
| PCaeC34:2 + C3 + His + Phe + Glu + | 0.982 | 0.02 |
| Fisher ratio | | |
| C3 + His + Phe + Glu + Fisher ratio + | 0.977 | 0.03 |
| Total PCae | | |
| PCaeC34:2 + C3 + His + Phe + Glu | 0.961 | 0.045 |
| PCaeC34:2 + C3 + His + Phe + Glu + Tyr | 0.950 | 0.05 |
| PCaeC34:2 + C3 + His + Phe + Glu + | 0.985 | |
| Hypoxanthine | | |
| PCaeC34:2 + C3 + His + Phe + Glu + | 0.994 | |
| Tyr + Hypoxanthine | | |
| Glu + Tyr/Phe + His + C0 + Total | 0.973 | 0.03 |
| PCae + Essential AA | | |
| Glu + Tyr/Phe + His + C0 + Total | 0.981 | 0.02 |
| PCae + Total ACOH/Total AC | | |
| Glu + Tyr/Phe + His + C0 + Total | 0.997 | 0.001 |
| PCae + Essential AA + Total ACOH/Total AC | | |

BNP: B-type natriuretic peptide, Essential AA: Essential amino acid; PCaeC34:2: Phosphatidylcholine acyl-alkyl C34:2, C3: Propionylcarnitine, His: Histidine, Pro: Proline, Glu: Glutamate, Tyr: Tyrosine, Phe: Phenylalanine, C0: Carnitine, Total ACOH: Total hydroxylacylcarnitine, Total PCae: Total phosphatidylcholine, Fisher Ratio: (Leucine + Isoleucine + Valine)/(Phenylalanine + Tryptophan + Tyrosine).
Total AC: Total acylcarnitine.

To discriminate patients at stage B from patients at stage A, a few good metabolite combinations were found as shown in the Table 7. The diagnosis values of these combinations are analyzed by ROC curves, and are presented by the area under the curve (AUC). The diagnostic values of metabolomics-derived parameters are better than BNP.

TABLE 7
Different combinations of global metabolites to discriminate
patients at stage B from patients at stage A

| | Area under | P value |
|---|---|---|
| | the curve | (compared |
| Metabolites | (AUC) | to BNP |
| BNP | 0.770 | — |
| PCaaC34:4 + Ala + SDMA + C5:1 | 0.883 | 0.004 |
| PCaaC34:4 + Ala + SDMA + C3 | 0.874 | 0.01 |
| PCaaC34:4 + Ala + SDMA + C5:1 + | 0.915 | |
| Hypoxanthine | | |
| PCaaC34:4 + Ala + SDMA + C3 + | 0.969 | |
| Hypoxanthine | | |
| PCaaC34:4 + Ala + SDMA + Sarcosine | 0.937 | 0.0001 |
| PCaaC34:4 + Ala + SDMA + Sarcosine | 0.982 | <0.0001 |

BNP: B-type natriuretic peptide, PCaaC34:4: Phosphatidylcholine diacyl C34:4, Ala: Alanine, SDMA: Symmetric dimethylarginine, C5:1: Tiglylcarnitine, C3: Propionylcarnitine.

To discriminate patients at stage B from patients at stage C, a few good metabolite combinations were found as shown in the Table 8. The diagnosis values of these combinations are analyzed by ROC curves, and are presented by the area under the curve (AUC). The diagnostic values of metabolomics-derived parameters are better than BNP.

TABLE 8
Different combinations of global metabolites to discriminate
patients at stage B from patients at stage C

| | Area under | P value |
|---|---|---|
| | the curve | (compared |
| Metabolites | (AUC) | to BNP) |
| BNP | 0.836 | — |
| Spermidine + PCaaC34:4 + PCaeC34:2 + | 0.892 | 0.01 |
| His + Fisher ratio | | |
| Spermidine + PCaeC34:2 + His + Fisher | 0.917 | 0.001 |
| ratio | | |
| Spermidine + His + Fisher ratio | 0.873 | 0.02 |
| Spermidine + PCaeC34:2 + His + Fisher | 0.939 | |
| ratio + Hypoxanthine | | |
| Spermidine + His + Fisher ratio + | 0.891 | |
| Hypoxanthine | | |
| Spermidine + SM C16:0 + C14:2 + Fisher | 0.924 | 0.001 |
| ratio | | |

BNP: B-type natriuretic peptide, PCaaC34:4: Phosphatidylcholine diacyl C34:4, PCaeC34:2: Phosphatidylcholine acyl-alkyl C34:2, His: Histidine, SM C16:0: Sphingomyelin C16:0, C14:2: Tetradecadienoylcarnitine, Fisher Ratio: a ratio of branched chain amino acids to aromatic amino acids.

5. Use of Metabolomics in Serial Estimations for Patients from Acute HF Status to Stabilized Status

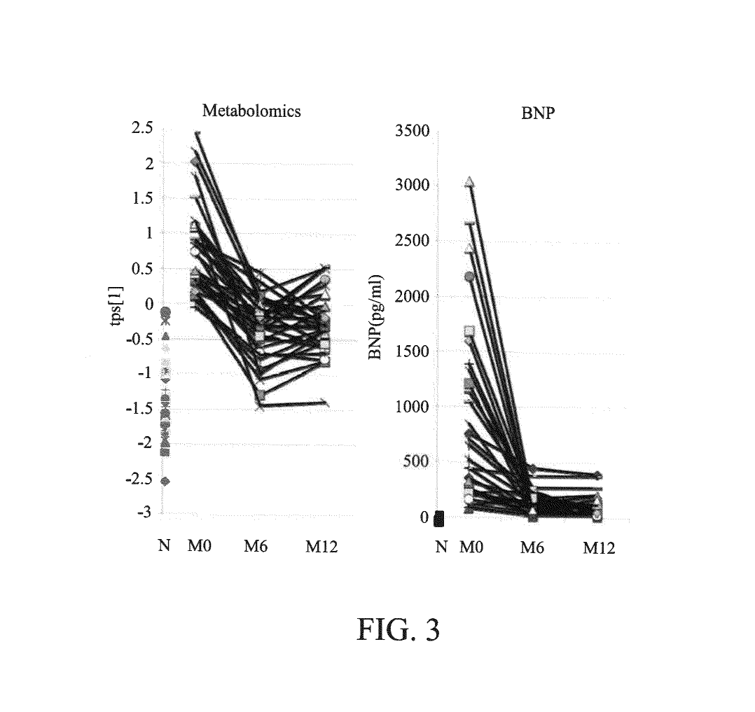

Based on the data described in table 5, it was tried to verify the diagnostic value of combining four metabolites (Histidine, Phenylalanine, Spermidine and Hypoxanthine). A parameter derived from the calculation of these 4 metabolites was produced, called tPS[1]. For this purpose, metabolomics analysis along with BNP measurement was further performed in 32 patients (22 males and 10 females, aged 54±11 years) at stage C. These patients were initially hospitalized due to acute cardiogenic pulmonary edema, got improved to NYHA functional classes I, and survived longer than one year. Plasma was analyzed before, and 6 and 12 months after discharged. The serial changes in tPS[ ] values were presented. As shown in FIG. 3, the tPS[1] values in the 32 patients at pre-discharge were significantly higher than the normal controls. Although the tPS[1] values remarkably decreased at 6 months, increases of tPS[1] were noted in part of the patients at 12 months. Compared to the levels at pre-discharge, BNP levels remarkably decreased at 6 months and remained steady at 12 months. These findings suggest that metabolomics analysis is a more sensitive tool than BNP in estimating HF status after acute HF

### Example 2

### Targeted Metabolomics Analyses for Diagnosing and Staging Heart Failure

### 1. Patients

A total of 145 subjects were enrolled in this example. This included 62 normal subjects and 83 patients at stage C.

### 2. Targeted Metabolomics Analyses in HF and Normal Controls

For the quantitation of metabolite concentrations, the Biocrates kit was applied in this example. Plasma was subjected to metabolomics analysis according to the targeted metabolomics workflow and datasets were bioinformatically analyzed using OPLS-DA model. To test whether these targeted metabolite profiles could discriminate stage C HF patients from normal controls, a total of 201 variables was used in the analysis. The metabolites responsible for the discrimination between these 2 groups (those metabolites with a VIP score>1.0) are listed in Table 9.

TABLE 9
Statistical analysis of targeted metabolites in the normal controls and patients at HF stage C

| Metabolite ID | | Normal control | Stage C | P-value |
|---|---|---|---|---|
| (μM) | | (n = 62) | (n = 83) | (t-test) |
| 1. | Histidine | 99.303 ± 18.043 | 77.736 ± 18.423 | 2.15E−09 |
| 2. | Phenylalanine | 57.437 ± 8.956 | 76.960 ± 22.607 | 1.42E−09 |
| 3. | Ornithine/Arginine | 1.027 ± 0.229 | 2.107 ± 1.320 | 1.51E−09 |
| 4. | Phosphatidylcholine diacyl C34:4 | 0.973 ± 0.362 | 0.596 ± 0.355 | 6.09E−08 |
| 5. | Ornithine | 56.921 ± 18.711 | 83.954 ± 32.530 | 4.50E−08 |
| 6. | Phosphatidylcholine diacyl C36:2 | 226.400 ± 58.442 | 172.928 ± 52.451 | 4.60E−07 |
| 7. | Octadecadienylcarnitine | 0.053 ± 0.034 | 0.105 ± 0.070 | 2.42E−07 |
| 8. | Phosphatidylcholine diacyl C36:1 | 44.837 ± 11.149 | 35.375 ± 10.244 | 3.24E−06 |
| 9. | Glutamate | 53.219 ± 43.497 | 147.612 ± 135.093 | 2.50E−07 |
| 10. | Phosphatidylcholine diacyl C36:0 | 4.981 ± 1.166 | 3.855 ± 1.435 | 9.09E−06 |
| 11. | Spermidine | 0.258 ± 0.034 | 0.372 ± 0.176 | 6.93E−07 |
| 12. | Spermine | 0.051 ± 0.161 | 0.301 ± 0.373 | 1.70E−06 |
| 13. | Phosphatidylcholine diacyl C40:5 | 9.322 ± 3.251 | 6.971 ± 2.600 | 1.79E−05 |
| 14. | Phosphatidylcholine diacyl C36:6 | 0.908 ± 0.468 | 0.562 ± 0.394 | 1.95E−05 |
| 15. | Phosphatidylcholine diacyl C33:3 | 0.231 ± 0.069 | 0.172 ± 0.079 | 0.00003 |
| 16. | Citrulline/Ornithine | 1.163 ± 0.737 | 0.677 ± 0.510 | 0.0001 |
| 17. | Phosphatidylcholine diacyl C38:5 | 44.186 ± 12.629 | 33.627 ± 14.051 | 3.60E−05 |
| 18. | Creatinine | 103.998 ± 41.635 | 185.324 ± 135.578 | 5.98E−06 |
| 19. | Phosphatidylcholine diacyl C36:3 | 94.046 ± 21.592 | 78.162 ± 21.183 | 8.19E−05 |
| 20. | Sphingomyeline C20:2 | 1.119 ± 0.298 | 0.873 ± 0.355 | 9.29E−05 |
| 21. | Phosphatidylcholine diacyl C38:3 | 31.133 ± 8.561 | 24.971 ± 8.234 | 9.59E−05 |
| 22. | Phosphatidylcholine acyl-alkyl C38:6 | 7.104 ± 2.281 | 5.519 ± 2.142 | 0.0001 |
| 23. | Putrescine/Ornithine | 0.008 ± 0.005 | 0.005 ± 0.003 | 0.0005 |
| 24. | Phosphatidylcholine acyl-alkyl C34:2 | 8.297 ± 2.463 | 6.568 ± 2.375 | 0.0001 |
| 25. | Spermine/Spermidine | 0.178 ± 0.564 | 0.677 ± 0.778 | 0.0001 |
| 26. | Aromatic amino acids | 163.971 ± 24.682 | 193.288 ± 49.833 | 3.34E−05 |
| 27. | Hydroxybutyrylcarnitine | 0.030 ± 0.015 | 0.049 ± 0.032 | 3.47E−05 |
| 28. | Phosphatidylcholine diacyl C40:4 | 2.758 ± 0.839 | 2.221 ± 0.711 | 0.0002 |
| 29. | Phosphatidylcholine acyl-alkyl C36:2 | 9.792 ± 2.232 | 8.096 ± 2.632 | 0.0003 |
| 30. | Hexose | 5284.765 ± 1986.055 | 6993.400 ± 2813.938 | 0.0001 |
| 31. | Phosphatidylcholine acyl-alkyl C36:3 | 6.427 ± 1.800 | 5.243 ± 1.712 | 0.0003 |
| 32. | Octadecenoylcarnitine | 0.122 ± 0.074 | 0.183 ± 0.102 | 0.0002 |
| 33. | Butrylcarnitine | 0.233 ± 0.065 | 0.352 ± 0.230 | 5.95E−05 |
| 34. | Phosphatidylcholine diacyl C36:5 | 18.999 ± 9.949 | 12.797 ± 9.264 | 0.0005 |
| 35. | Phosphatidylcholine acyl-alkyl C36:4 | 12.616 ± 4.188 | 10.299 ± 3.158 | 0.0006 |
| 36. | Phosphatidylcholine acyl-alkyl C34:3 | 5.202 ± 1.929 | 4.079 ± 1.721 | 0.0009 |
| 37. | Glutamine | 733.303 ± 134.001 | 624.401 ± 199.760 | 0.0009 |
| 38. | Fisher ratio | 2.878 ± 0.419 | 2.502 ± 0.711 | 0.0003 |
| 39. | Phosphatidylcholine acyl-alkyl C40:1 | 1.319 ± 0.362 | 1.065 ± 0.445 | 0.0010 |
| 40. | Phosphatidylcholine diacyl C28:1 | 1.402 ± 0.324 | 1.169 ± 0.418 | 0.0011 |
| 41. | Phosphatidylcholine acyl-alkyl C36:5 | 9.951 ± 3.435 | 8.114 ± 2.781 | 0.0021 |
| 42. | Total Phosphatidylcholine acyl-alkyl | 129.041 ± 28.626 | 111.007 ± 32.806 | 0.0019 |
| 43. | Decanoylcarnitine | 0.278 ± 0.117 | 0.218 ± 0.094 | 0.0020 |
| 44. | Phosphatidylcholine diacyl C42:6 | 0.545 ± 0.152 | 0.465 ± 0.135 | 0.0024 |
| 45. | Tyrosine | 67.599 ± 12.932 | 79.768 ± 25.909 | 0.0008 |
| 46. | Taurine | 44.966 ± 8.465 | 58.678 ± 31.475 | 0.0006 |
| 47. | Phosphatidylcholine diacyl C34:3 | 10.689 ± 3.391 | 8.680 ± 3.807 | 0.0031 |
| 48. | Phosphatidylcholine diacyl C38:0 | 3.821 ± 1.190 | 3.131 ± 1.298 | 0.0031 |
| 49. | Phosphatidylcholine diacyl C42:5 | 0.282 ± 0.100 | 0.228 ± 0.098 | 0.0032 |
| 50. | Phosphatidylcholine diacyl C38:4 | 81.812 ± 22.096 | 68.326 ± 26.379 | 0.0034 |
| 51. | Phosphatidylcholine acyl-alkyl C30:0 | 0.272 ± 0.075 | 0.230 ± 0.080 | 0.0034 |
| 52. | Phosphatidylcholine acyl-alkyl C30:1 | 0.308 ± 0.068 | 0.266 ± 0.087 | 0.0044 |
| 53. | Phosphatidylcholine diacyl C40:6 | 28.904 ± 11.144 | 23.258 ± 10.341 | 0.0045 |
| 54. | Phosphatidylcholine acyl-alkyl C38:1 | 3.324 ± 0.671 | 2.856 ± 1.014 | 0.0047 |
| 55. | Phosphatidylcholine acyl-alkyl C44:5 | 0.922 ± 0.398 | 1.119 ± 0.359 | 0.0047 |
| 56. | Phosphatidylcholine acyl-alkyl C40:6 | 3.805 ± 0.986 | 3.223 ± 1.204 | 0.0051 |
| 57. | Phosphatidylcholine diacyl C34:2 | 398.289 ± 93.260 | 347.433 ± 101.429 | 0.0053 |
| 58. | Isoleucine | 72.689 ± 17.269 | 84.387 ± 26.271 | 0.0062 |
| 59. | DMA/Arginine | 0.004 ± 0.011 | 0.013 ± 0.020 | 0.0028 |
| 60. | Phosphatidylcholine diacyl C32:0 | 9.482 ± 1.963 | 10.622 ± 2.477 | 0.0071 |
| 61. | Valerycarnitine | 0.101 ± 0.036 | 0.149 ± 0.121 | 0.0021 |
| 62. | Phosphatidylcholine acyl-alkyl C38:0 | 2.071 ± 1.106 | 1.508 ± 1.170 | 0.0080 |
| 63. | Glycine | 225.479 ± 58.069 | 261.766 ± 84.559 | 0.0054 |
| 64. | Octanoylcarnitine | 0.197 ± 0.079 | 0.165 ± 0.056 | 0.0142 |
| 65. | Tetradecenoylcarnitine | 0.084 ± 0.057 | 0.110 ± 0.050 | 0.0097 |
| 66. | Phosphatidylcholine diacyl C38:6 | 81.443 ± 25.515 | 67.862 ± 31.996 | 0.0130 |
| 67. | Phosphatidylcholine acyl-alkyl C32:2 | 0.419 ± 0.140 | 0.356 ± 0.140 | 0.0151 |
| 68. | Phosphatidylcholine diacyl C30:0 | 2.134 ± 0.537 | 1.907 ± 0.496 | 0.0170 |
| 69. | Phosphatidylcholine acyl-alkyl C38:4 | 9.389 ± 2.402 | 8.347 ± 2.350 | 0.0176 |
| 70. | Phosphatidylcholine acyl-alkyl C38:2 | 3.909 ± 0.808 | 3.415 ± 1.326 | 0.0195 |
| 71. | Phosphatidylcholine acyl-alkyl C38:5 | 12.521 ± 3.380 | 11.122 ± 3.174 | 0.0203 |
| 72. | Spermidine/Putrescine | 0.731 ± 0.530 | 1.081 ± 0.917 | 0.0226 |
| 73. | Phosphatidylcholine diacyl C36:4 | 135.325 ± 38.086 | 117.295 ± 46.025 | 0.0231 |
| 74. | Arginine | 56.363 ± 18.312 | 48.226 ± 20.136 | 0.0212 |
| 75. | Propionylcarnitine | 0.357 ± 0.108 | 0.447 ± 0.268 | 0.0116 |
| 76. | DMA | 0.199 ± 0.494 | 0.452 ± 0.705 | 0.0203 |
| 77. | Phosphatidylcholine acyl-alkyl C44:6 | 0.549 ± 0.265 | 0.641 ± 0.199 | 0.0294 |
| 78. | Phosphatidylcholine diacyl C40:3 | 0.622 ± 0.192 | 0.534 ± 0.240 | 0.0308 |
| 79. | Hexadecanoylcarnitine | 0.087 ± 0.043 | 0.112 ± 0.071 | 0.0199 |
| 80. | LysoPhosphatidylcholine acyl C14:0 | 2.774 ± 0.565 | 2.551 ± 0.578 | 0.0354 |
| 81. | Alpha-Aminoadipic acid | 1.009 ± 0.664 | 1.263 ± 0.654 | 0.0365 |
| 82. | Hydroxysphingomyeline C14:1 | 4.212 ± 0.847 | 3.802 ± 1.205 | 0.0282 |
| 83. | Phosphatidylcholine acyl-alkyl C38:3 | 3.713 ± 1.112 | 3.248 ± 1.363 | 0.0462 |
| 84. | Phosphatidylcholine acyl-alkyl C36:1 | 6.186 ± 1.336 | 5.589 ± 1.817 | 0.0483 |
| 85. | Asymmetric dimethylarginine/Arginine | 0.000 ± 0.001 | 0.011 ± 0.037 | 0.0207 |
| 86. | Asymmetric dimethylarginine | 0.028 ± 0.116 | 0.369 ± 1.236 | 0.0222 |
| 87. | Glucogenic amino acids | 717.168 ± 109.617 | 784.870 ± 237.345 | 0.0352 |
| 88. | Symmetric dimethylarginine/Arginine | 0.004 ± 0.011 | 0.010 ± 0.019 | 0.0433 |
| 89. | Symmetric dimethylarginine | 0.128 ± 0.327 | 0.267 ± 0.461 | 0.0524 |
| 90. | Proline | 166.166 ± 61.575 | 189.780 ± 78.301 | 0.0745 |
| 91. | L-Dopa | 0.173 ± 0.264 | 0.099. ± 0.210 | 0.0963 |
| 92. | Decadienylcarnitine | 0.399 ± 0.126 | 0.342 ± 0.232 | 0.0836 |
| 93. | Valine | 250.655 ± 56.145 | 234.922 ± 70.229 | 0.1861 |
| 94. | Tryptophan | 38.935 ± 9.790 | 36.560 ± 11.643 | 0.2359 |
| 95. | Tiglylcarnitine | 0.032 ± 0.019 | 0.036 ± 0.023 | 0.2839 |
| 96. | Citrulline | 58.040 ± 27.607 | 53.524 ± 41.084 | 0.4656 |
| 97. | Putrescine | 0.430 ± 0.256 | 0.404 ± 0.228 | 0.5499 |
| 98. | Leucine | 149.621 ± 44.640 | 147.630 ± 47.565 | 0.8145 |
| 99. | Threonine | 111.582 ± 20.515 | 112.500 ± 33.305 | 0.8500 |
| 100. | Lysine | 207.936 ± 47.620 | 209.698 ± 66.436 | 0.8712 |

Data are presented as mean ± SD.
DMA: DMA represents total dimethylarginine

### 3. Discrimination of Patients at Stage C and Normal Controls

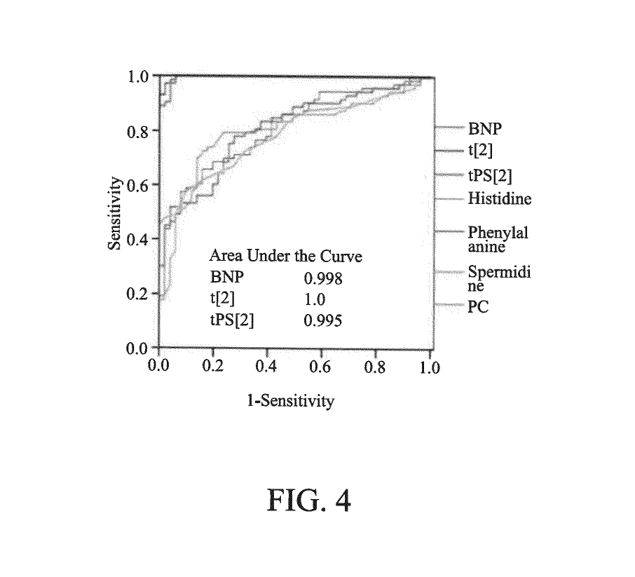

To discriminate patients at stage C and normal controls (diagnostic value), the ROC curves were drawn for both BNP and t[2](by taking all targeted metabolites into account by using the principal component analysis) (FIG. 4). The areas under the curves were 0.998 and 1.0, respectively. In the targeted metabolomics data set, it was found four important metabolites that significantly contributed to the diagnostic value of metabolomics for HF, including histidine, phenylalanine, spermidine, and phosphatidylcholine diacyl C34:4 (Table 10). A combination of these 4 metabolites reached an area under curve of 0.995, which was better than each metabolite alone (FIG. 4). Based on the combination of these 4 metabolites, a parameter was produced, called tPS[2]. The diagnostic values of the BNP and tPS[2] levels for identifying stage C HF (vs. normal controls) were presented in Table 10.

TABLE 10
Diagnostic values of BNP and targeted metabolites in patients
with heart failure at stage C (versus normal controls)

| | Area under | P value |
|---|---|---|
| | the curve | (compared to |
| | (AUC) | BNP) |
| BNP | 0.998 | — |
| t[2](all targeted metabolites) | 1.000 | 0.92 |
| Histidine | 0.752 | <0.001 |
| Phenylalanine | 0.733 | <0.001 |
| PC aa C34:4 | 0.715 | <0.001 |
| Spermidine | 0.712 | <0.001 |
| tPS[2] (Four metabolites: | 0.995 | 0.97 |
| Histidine, Phenylalanine, PC | | |
| aa C34:4, Spermidine) | | |

BNP, B-type natriuretic peptide level; PC aa, phosphatidylcholine diacyl.
“t[2]” is a parameter derived from the whole targeted metabolites dataset; “tPS[2]” is a parameter derived from the 4 metabolites: Histidine, Phenylalanine, PC aa C34:4 (Phosphatidylcholine diacyl C34:4) and Spermidine.

### Example 3

### Targeted Metabolomics Analyses for Evaluating a Prognosis for Heart Failure

### 1. Prognostic Values of Metabolomic Signature

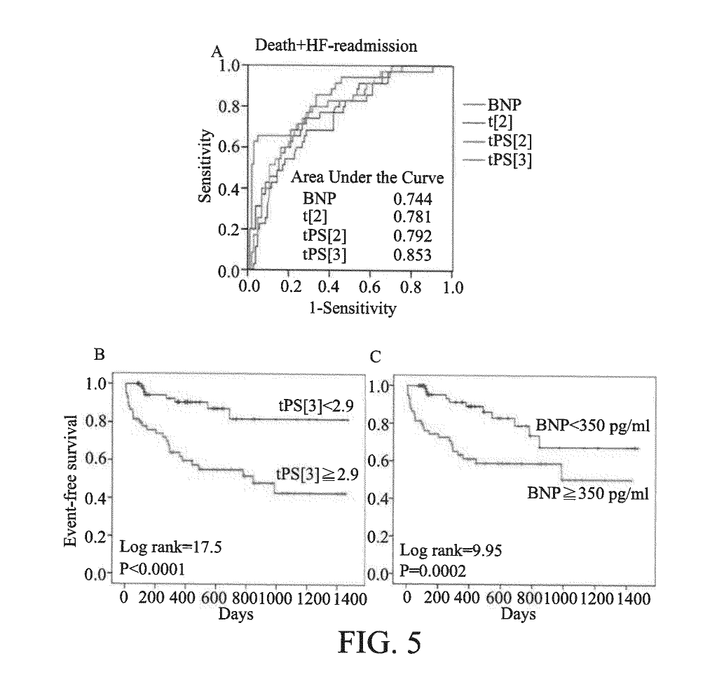

To estimate the prognostic values of metabolomics and BNP, the following analyses focused on patients at stages B and C. To look for potential metabolic predictors of a composite event of all-cause death and HF-related re-hospitalization, extensive analyses on the targeted metabolite dataset revealed that a combination of 4 classes of metabolites (Dimethylarginine/Arginine ratio, spermidine, butyrylcarnitine, and total amount of essential amino acids) gave rise to an optimal prognostic value remarkably better than BNP. By combining these 4 classes of metabolites, a parameter was produced, called tPS[3]. The AUC of ROC curves were 0.853, 0.792, and 0.744, respectively to tPS[3], tPS[2](derived from the whole targeted metabolomics dataset), and BNP levels (FIG. 5A). Table 11 showed the data of AUC (by ROC curves) and Log Rank values (by Kaplan-Meier analysis) for these parameters on prognosis.

The mean of tPS[3](2.9, range 0.04-5.63) was set as the cutoff value for prognostic prediction. In FIG. 5B, the Kaplan-Meier curves demonstrated that a tPS[3] of ≧2.9 at pre-discharge was associated with a higher composite event rate of HF-related re-hospitalization and all-cause death (Log rank=17.5, p<0.0001). In comparison, the prognostic values of a BNP of ≧350 pg/ml is shown in FIG. 5C (Log rank=9.9, p=0.002).

TABLE 11
Prognostic values of BNP and targeted metabolites
in patients with heart failure

| | | | P value |
|---|---|---|---|
| | Area under the | Log Rank | (Compared |
| | curve (by ROC | (by Kaplan- | to BNP by |
| | curve) | Meire) | chi-square) |
| BNP | 0.744 | 9.95 | — |
| t[2] | 0.781 | 10.7 | 0.04 |
| tPS[2] | 0.792 | 12.1 | <0.001 |
| DMA/Arginine ratio | 0.730 | 8.8 | 0.06 |
| Spermidine | 0.712 | 8.2 | 0.042 |
| Butyrylcarnitine | 0.698 | 7.65 | 0.04 |
| Total essential amino acids | 0.674 | 7.33 | 0.03 |
| tPS[3] (Four metabolites: | 0.853 | 17.5 | <0.0001 |
| DMA/Arginine ratio, | | | |
| Spermidine, | | | |
| butyrylcarnitine, Total | | | |
| essential amino acids) | | | |

BNP represents B-type natriuretic peptide level; t[2] is a parameter derived from all targeted metabolomics analysis; tPS[2] is a parameter derived from 4 targeted metabolites (Histidine, Phenylalanine, PC aa C34:4 (phosphatidylcholine diacyl C34:4), Spermidine); DMA represents total dimethylarginine.
The essential amino acids comprise phenylalanine, valine, threonine, tryptophan, isoleucine, leucine, methionine, lysine and histidine.

### Example 4

### The Prognostic Values of Global Metabolites Analyses for the Patients with Heart Failure

Global metabolomics analysis was performed in this example. A total of 157 patients were enrolled, including patients at stages B (n=81), and C (n=76). A global metabolomics analysis was used to identify different combinations of metabolites with good values on predicting a composite event of death and heart failure-related re-hospitalization.

For the prognostic values of metabolomics and BNP, the estimations were based on AUC (derived from ROC curves), and “Log Rank” values (derived from Kaplan-Meier analysis). The data were shown in Table 12.

### 1. Comparisons of BNP and Different Combinations of Global Metabolites on Prognostic Values:

(1). By AUC (Derived from ROC Curves):

Initially, it was found that the prognostic values of metabolomics is better than BNP while combining 4 classes of metabolites, including dimethylarginine/arginine, butyrylcarnitine, spermidine, and total essential amino acid.

A combination of dimethylarginine/arginine and butyrylcarnitine is already better than BNP based on Table 12. A combination of dimethylarginine/arginine, butyrylcarnitine, and spermidine is already better than BNP. A combination of dimethylarginine/arginine, butyrylcarnitine, and xanthine is already better than BNP. A combination of dimethylarginine/arginine and xanthine is already better than BNP. A combination of dimethylarginine/arginine, xanthine, and tryptophan is already better than BNP. A combination of dimethylarginine/arginine, xanthine, and spermidine/spermine is already better than BNP. Xanthine alone is already better than BNP. A combination of SDMA (symmetric dimethylarginine)/arginine and xanthine is already better than BNP. A combination of SDMA/arginine, xanthine, and tryptophan is already better than BNP. A combination of SDMA/arginine, xanthine, and spermidine/spermine is already better than BNP. SDMA alone is already better than BNP. SDMA/arginine alone is already better than BNP. P-cresyl sulfate alone is already better than BNP. A combination of SDMA, and P-cresyl sulfate is already better than BNP. A combination of SDMA, P-cresyl sulfate, and Phosphatidylcholine diacyl C38:6 is already better than BNP. A combination of SDMA, P-cresyl sulfate, and butyrylcarnitine is already better than BNP. A combination of SDMA, P-cresyl sulfate, and spermidine is already better than BNP. A combination of DMA/arginine, and P-cresyl sulfate is already better than BNP. A combination of DMA/arginine, P-cresyl sulfate, and Phosphatidylcholine diacyl C38:6 is already better than BNP. A combination of DMA/arginine, P-cresyl sulfate, and butyrylcarnitine is already better than BNP. A combination of DMA/arginine, P-cresyl sulfate, and spermidine is already better than BNP. A combination of dimethylarginine/arginine and spermidine is already better than BNP. A combination of SDMA/arginine and spermidine is already better than BNP. A combination of SDMA/arginine and butyrylcarnitine is already better than BNP. A combination of tryptophan and xanthine is already better than BNP. A combination of tryptophan and spermidine is already better than BNP. A combination of tryptophan and butyrylcarnitine is already better than BNP. A combination of leucine and xanthine is already better than BNP. A combination of leucine and spermidine is already better than BNP. A combination of leucine and butyrylcarnitine is already better than BNP. A combination of threonine and xanthine is already better than BNP. A combination of threonine and spermidine is already better than BNP. A combination of threonine and butyrylcarnitine is already better than BNP.

However, it was noted that the dimethylarginine/arginine only is worse than BNP.

(2). By “Log Rank” Value (Derived from Kaplan-Meier Analysis): (the Cutoff is Set at the Mean Value of Each Parameter)

Initially, it was found that the prognostic value of metabolomics is better than BNP while combining 4 classes of metabolites, including dimethylarginine/arginine, butyrylcarnitine, spermidine, and total essential amino acid.

A combination of dimethylarginine/arginine and butyrylcarnitine is already better than BNP. A combination of dimethylarginine/arginine, butyrylcarnitine, and spermidine is already better than BNP. A combination of dimethylarginine/arginine, butyrylcarnitine, and xanthine is already better than BNP. The dimethylarginine/arginine only is still better than BNP. A combination of dimethylarginine/arginine and xanthine is already better than BNP. A combination of dimethylarginine/arginine, xanthine, and tryptophan is already better than BNP. A combination of dimethylarginine/arginine, xanthine, and spermidine/spermine is already better than BNP. Xanthine alone is already better than BNP. A combination of SDMA (symmetric dimethylarginine)/arginine, and xanthine is already better than BNP. A combination of SDMA/arginine, xanthine, and tryptophan is already better than BNP. A combination of SDMA/arginine, xanthine, and spermidine/spermine is already better than BNP. SDMA alone is already better than BNP. SDMA/arginine alone is already better than BNP. P-cresyl sulfate alone is already better than BNP. A combination of SDMA, and P-cresyl sulfate is already better than BNP. A combination of SDMA, P-cresyl sulfate, and Phosphatidylcholine diacyl C38:6 is already better than BNP. A combination of SDMA, P-cresyl sulfate, and butyrylcarnitine is already better than BNP. A combination of SDMA, P-cresyl sulfate, and spermidine is already better than BNP. A combination of DMA/arginine, and P-cresyl sulfate is already better than BNP. A combination of DMA/arginine, P-cresyl sulfate, and Phosphatidylcholine diacyl C38:6 is already better than BNP. A combination of DMA/arginine, P-cresyl sulfate, and butyrylcarnitine is already better than BNP. A combination of DMA/arginine, P-cresyl sulfate, and spermidine is already better than BNP. A combination of dimethylarginine/arginine and spermidine is already better than BNP. A combination of SDMA/arginine and spermidine is already better than BNP. A combination of SDMA/arginine and butyrylcarnitine is already better than BNP. A combination of tryptophan and xanthine is already better than BNP. A combination of tryptophan and spermidine is already better than BNP. A combination of tryptophan and butyrylcarnitine is already better than BNP. A combination of leucine and xanthine is already better than BNP. A combination of leucine and spermidine is already better than BNP. A combination of leucine and butyrylcarnitine is already better than BNP. A combination of threonine and xanthine is already better than BNP. A combination of threonine and spermidine is already better than BNP. A combination of threonine and butyrylcarnitine is already better than BNP.

### (3). Replacement of the Total Essential Amino Acids by 2 or 3 Essential Amino Acids:

For evaluating the prognosis of heart failure, when the total essential amino acid was used in the combinations of metabolites as described above, it was noted that the total essential amino acids (9 amino acids) could be replaced by using only three amino acids (leucine, threonine and tryptophan) with the similar prognostic values. Furthermore, it was noted that the total essential amino acids (9 amino acids) could also be replaced by using only two amino acids (leucine and threonine, or leucine and tryptophan) with the similar prognostic values. (see Table 12)

TABLE 12
Comparisons of different combinations of global metabolites to B-type natriuretic
peptide on the prognostic values in patients with heart failure

| | | | P value |
|---|---|---|---|
| | Area under the | Log Rank | (Compared |
| | curve (by ROC | (by Kaplan- | to BNP by |
| Biomarkers | curve) | Meire) | chi-square) |
| B-type natriuretic peptide (BNP) | 0.744 | 9.95 | — |
| Metabolites from global | | | |
| metabolomics analysis | | | |
| DMA/Arginine, Butyrylcarnitine, and | 0.971 | 23.7 | <0.0001 |
| Xanthine | | | |
| DMA/Arginine and Xanthine | 0.966 | 22.5 | <0.0001 |
| DMA/Arginine, Xanthine, and Tryptophan | 0.973 | 23.1 | <0.0001 |
| DMA/Arginine, Xanthine, and | 0.972 | 23.9 | <0.0001 |
| Spermidine/Spermine | | | |
| Xanthine | 0.902 | 19.7 | <0.0001 |
| SDMA/Arginine, Xanthine, and Tryptophan | 0.974 | 24.0 | <0.0001 |
| SDMA, Xanthine, and Tryptophan | 0.972 | 23.8 | <0.0001 |
| SDMA/Arginine, and Xanthine | 0.969 | 22.4 | <0.0001 |
| SDMA/Arginine, Xanthine, and | 0.970 | 22.9 | <0.0001 |
| Spermidine/Spermine | | | |
| SDMA | 0.861 | 18.2 | <0.01 |
| SDMA/Arginine | 0.863 | 18.3 | <0.01 |
| P-cresyl sulfate | 0.766 | 10.0 | 0.04 |
| SDMA, and P-cresyl sulfate | 0.975 | 24.2 | <0.0001 |
| SDMA, P-cresyl sulfate, and PCaaC38:6 | 0.981 | 25.5 | <0.0001 |
| SDMA, P-cresyl sulfate, and Butyrylcarnitine | 0.977 | 24.7 | <0.0001 |
| SDMA, P-cresyl sulfate, and spermidine | 0.979 | 24.9 | <0.0001 |
| DMA/Arginine, and P-cresyl sulfate | 0.965 | 22.1 | <0.0001 |
| DMA/Arginine, P-cresyl sulfate, and | 0.972 | 24.0 | <0.0001 |
| PCaaC38:6 | | | |
| DMA/Arginine, P-cresyl sulfate, and | 0.966 | 22.3 | <0.0001 |
| Butyrylcarnitine | | | |
| DMA/Arginine, P-cresyl sulfate, and | 0.961 | 20.3 | <0.0001 |
| spermidine | | | |
| DMA/Arginine, Butyrylcarnitine, Spermidine, | 0.912 | 20.3 | <0.0001 |
| and Total essential amino acid | | | |
| DMA/Arginine, Butyrylcarnitine, Spermidine, | 0.952 | 18.3 | <0.0001 |
| Leucine, Threonine, and Tryptophan | | | |
| DMA/Arginine, Butyrylcarnitine, Spermidine, | 0.969 | 19.7 | <0.0001 |
| Leucine, and Threonine | | | |
| DMA/Arginine, Butyrylcarnitine, Spermidine, | 0.963 | 19.1 | <0.0001 |
| Leucine, and Tryptophan | | | |
| DMA/Arginine, Butyrylcarnitine, and | 0.951 | 15.4 | <0.0001 |
| Spermidine | | | |
| DMA/Arginine and Butyrylcarnitine | 0.947 | 15.1 | <0.0001 |
| DMA/Arginine | 0.757 | 9.7 | 0.11 |
| DMA/Arginine, Butyrylcarnitine, and | 0.940 | 13.2 | <0.0001 |
| PCaaC38:6 | | | |
| DMA/Arginine + Spermidine | 0.861 | 18.1 | 0.0001 |
| SDMA/Arginine + Spermidine | 0.874 | 18.9 | 0.0001 |
| SDMA/Arginine + Butyrylcarnitine | 0.936 | 21.5 | 0.0001 |
| SDMA/Arginine + Xanthine | 0.970 | 22.1 | 0.0001 |
| Tryptophan + Xanthine | 0.825 | 15.1 | 0.006 |
| Tryptophan + Spermidine | 0.813 | 14.4 | 0.012 |
| Tryptophan + Butyrylcarnitine | 0.775 | 10.1 | 0.032 |
| Leucine + Xanthine | 0.782 | 10.3 | 0.003 |
| Leucine + Spermidine | 0.755 | 9.98 | 0.137 |
| Leucine + Butyrylcarnitine | 0.754 | 9.97 | 0.094 |
| Threonine + Xanthine | 0.820 | 14.7 | 0.006 |
| Threonine + Spermidine | 0.817 | 14.2 | 0.087 |
| Threonine + Butyrylcarnitine | 0.849 | 16.5 | 0.109 |

DMA: Total dimethylarginine,
SDMA: Symmetric dimethylarginine,
PCaaC38:6: Phosphatidylcholine diacyl C38:6

### Example 5

### Kits for Diagnosing Heart Failure

### 1. Sample Extraction

### (1). Plasma Sample Preparation for Global Metabolites Analysis

To 100 μl of plasma, 400 μl acetonitrile (ACN) is added. The mixture will be vortexed for 30 s, sonicated for 15 min. and centrifuged at 10,000×g for 25 min. The supernatant will be collected into a separate tube. The pellets will be re-extracted once. To the residual pellets, an equivalent volume of aqueous methanol (1:1 methanol/water, volume to volume) will be added. The suspension will be vortexed for 30 s, sonicated for 15 min and again centrifuged to remove the precipitates. The aqueous methanolic supernatant and acetonitrile supernatant will be pooled and dried in a nitrogen evaporator. The residues will be saved and stored at −80° C. Residues can be suspended in 100 μl of 95:5 water/acetonitrile and centrifuged at 14,000×g for 5 min, the clear supernatant will be collected a for LC-MS analysis.

### (2). Plasma Sample Preparation for Lipid Analysis

For extraction of lipids, a modification of Folch's method will be employed.

Briefly stated, the 100 μl plasma will be transferred to a glass tube. Six milliliters of chloroform/methanol (2:1, v/v) solution and 1.5 ml of water are added. The sample will be vortexed 4 times for 30 s, and subsequently centrifuged at 700×g for 30 min at 4□. The upper phase is removed as completely as possible, and the lower phase is sonicated for 10 min. The sample will be centrifuged at 700×g for 10 min at 4□. The upper phase can be removed as completely as possible, and the lower phase was allowed to stand still at 4□:. Three milliliters of this sample will be dried under nitrogen gas, and stored at −80□. Prior to analysis, the sample will be dissolved in 200 μl of 40% methanol.

### 2. Metabolites Identification by Diagnostic Device

### (1). MS/MS Analyses

For structural identification of target metabolite, standards will be operated under identical chromatographic conditions with that of the profiling experiment. MS and MS/MS analyses are performed in the same conditions. MS/MS spectra are collected at 0.1 spectra per second, with a medium isolation window of ˜4 m/z. The collision energy will be set from 5 to 35 V. Several metabolites will be further confirmed by an ion mobility mass spectrometer under similar chromatographic conditions.

### (2). Fluorescence Spectroscopy

The concentration of histidine (or other metabolites, such as xanthine, spermidine, propionylcarnitine, butyrylcarnitine, P-cresyl sulfate and a combination thereof) in plasma will be determined with a method in which histidine (or other metabolites, such as xanthine, spermidine, propionylcarnitine, butyrylcarnitine, P-cresyl sulfate and a combination thereof) and o-phthaldialdehyde react in alkali to form a fluorescent product which is measured in a fluorescence spectrometer. The method is linear in the range used.

A diagnostic device used herein is not limited to the above examples. Based on the nature of a metabolite, other diagnostic device, such as biochip, ELISA, LC-MS, etc. can also be employed for detecting the metabolites identified herein.

Metabolomics analysis explores the global metabolic abnormalities in patients with heart failure. By using metabolomics analysis, this patent provides information associated with heart failure more than BNP and traditional markers provide. Analysis of the abundant metabolites in plasma explored the global sophisticated metabolic perturbation behind an abnormal BNP level, including up-regulation of glutamate-ornithine-proline, polyamine, purine and taurine synthesis pathways; down-regulation of nitric oxide, dopamine, and phosphatidylcholines synthesis pathways during progression of HF (see FIG. 2) and changes in urea cycle, biopterin cycle, MTA cycle, methionine cycle, ornithine-proline-glutamate, polyamine synthesis, dopamine synthesis, methylation (creatinine and phosphatidylcholine), transsulfuration (taurine) and purine metabolism. The plasma concentrations of a few metabolites (for example, xanthine, histidine, phenylalanine, ornithine, arginine, spermine, spermidine, taurine, and phosphatidylcholines) changed at different stages of HF, and these metabolites changes are potential biomarkers.

By using metabolomics analysis, this patent provides more sensitive and specific metabolic evaluation for HF staging than ACC/AHA classification, BNP and other traditional markers provide. The methods provided in this patent are able to discriminate patients at HF stage C from the healthy subjects, patients at HF stage A from the healthy subjects, and patients at HF stage C from patients at HF stage A. By the methods provided in this patent, the discrimination among patients at different HF stages is more scientific than the way by ACC/AHA classification.

By using metabolomics analysis, this patent identifies novel biomarkers (for example, by combining xanthine, spermidine, butyrylcarnitine, some phosphatidylcholines, and other metabolites) to provide better diagnostic and prognostic values for patients with heart failure than BNP and traditional markers provide.

While some of the embodiments of the present invention have been described in detail in the above, it is, however, possible for those of ordinary skill in the art to make various modifications and changes to the particular embodiments shown without substantially departing from the teaching and advantages of the present invention. Such modifications and changes are encompassed in the spirit and scope of the present invention as set forth in the appended claim.

What is claimed is:

1. A method for diagnosing heart failure in a subject, comprising steps of:
measuring a biological sample of the subject to obtain an amount of at least one biomarker selected from the group consisting of xanthine, spermidine, propionylcarnitine, butyrylcarnitine and P-cresyl sulfate; and
comparing the amount of the at least one biomarker to a reference,

2. The method according to claim 1, wherein the biological sample is selected from the group consisting of blood, plasma, serum and urine.

3. The method of claim 1, further comprising steps of measuring a biological sample of the subject to obtain an amount of amino acid; and comparing the amount of the amino acid to a reference of the amino acid.

4. The method according to claim 3, wherein the amino acid is selected from the group consisting of glutamine, tyrosine, phenylalanine, histidine, arginine, leucine, tryptophan, threonine, isoleucine, lysine, methionine, valine and proline.

5. The method of claim 1, further comprising steps of measuring a biological sample of the subject to obtain an amount of hypoxanthine; and comparing the amount of the hypoxanthine to a reference of the hypoxanthine.

6. The method of claim 1, further comprising steps of measuring a biological sample of the subject to obtain an amount of phosphatidylcholine; and comparing the amount of the phosphatidylcholine to a reference, wherein the phosphatidylcholine is selected from the group consisting of phosphatidylcholine diacyl C34:4, phosphatidylcholine acyl-alkyl C36:2, phosphatidylcholine acyl-alkyl C34:2, phosphatidylcholine acyl-alkyl C34:3, phosphatidylcholine diacyl C36:0, phosphatidylcholine diacyl C36:1, phosphatidylcholine diacyl C36:3, phosphatidylcholine diacyl C38:6, phosphatidylcholine diacyl C36:6, phosphatidylcholine diacyl C38:5, phosphatidylcholine diacyl C40:5, phosphatidylcholine diacyl C36:2, phosphatidylcholine acyl-alkyl C36:5, phosphatidylcholine diacyl C38:0, phosphatidylcholine acyl-alkyl C32:3, phosphatidylcholine diacyl C40:4, phosphatidylcholine acyl-alkyl C38:3 and phosphatidylcholine diacyl C42:6.

7. The method according to claim 6, the phosphatidylcholine is selected from the group consisting of phosphatidylcholine acyl-alkyl C34:2, phosphatidylcholine acyl-alkyl C34:3 and phosphatidylcholine diacyl C34:4.

8. A method for staging heart failure in a subject, comprising steps of:
measuring a biological sample of the subject to obtain an amount of at least one biomarker selected from the group consisting of xanthine, spermidine and propionylcarnitine; and
comparing the amount of the at least one biomarker to a reference.

9. The method according to claim 8, further comprising steps of measuring a biological sample of the subject to obtain an amount of amino acid; and comparing the amount of the amino acid to a reference of the amino acid.

10. The method according to claim 9, wherein the amino acid is selected from the group consisting of glutamine, tyrosine, phenylalanine, histidine, arginine, leucine, tryptophan, threonine, isoleucine, lysine, methionine, valine and proline.

11. The method of claim 8, further comprising steps of measuring a biological sample of the subject to obtain an amount of hypoxanthine; and comparing the amount of the hypoxanthine to a reference of the hypoxanthine.

12. The method of claim 8, further comprising steps of measuring a biological sample of the subject to obtain an amount of phosphatidylcholine; and comparing the amount of the phosphatidylcholine to a reference of the phosphatidylcholine, wherein the phosphatidylcholine is selected from the group consisting of phosphatidylcholine diacyl C34:4, phosphatidylcholine acyl-alkyl C36:2, phosphatidylcholine acyl-alkyl C34:2, phosphatidylcholine acyl-alkyl C34:3, phosphatidylcholine diacyl C36:0, phosphatidylcholine diacyl C36:1, phosphatidylcholine diacyl C36:3, phosphatidylcholine diacyl C38:6, phosphatidylcholine diacyl C36:6, phosphatidylcholine diacyl C38:5, phosphatidylcholine diacyl C40:5, phosphatidylcholine diacyl C36:2, phosphatidylcholine acyl-alkyl C36:5, phosphatidylcholine diacyl C38:0, phosphatidylcholine acyl-alkyl C32:3, phosphatidylcholine diacyl C40:4, phosphatidylcholine acyl-alkyl C38:3 and phosphatidylcholine diacyl C42:6.

13. The method according to claim 12, the phosphatidylcholine is selected from the group consisting of phosphatidylcholine acyl-alkyl C34:2, phosphatidylcholine acyl-alkyl C34:3 and phosphatidylcholine diacyl C34:4.

14. A method for evaluating a prognosis of heart failure in a subject, comprising steps of:
measuring a biological sample of the subject to obtain an amount of at least one biomarker selected from the group consisting of xanthine, spermidine, butyrylcarnitine and P-cresyl sulfate; and
comparing the amount of the at least one biomarker to a reference.

15. The method according to claim 14, further comprising steps of measuring a biological sample of the subject to obtain an amount of amino acid; and comparing the amount of the amino acid to a reference of the amino acid.

16. The method according to claim 15, wherein the amino acid is an essential amino acid.

17. The method according to claim 16, wherein the essential amino acid is selected from the group consisting of histidine, isoleucine, leucine, lysine, methionine, phenylalanine, threonine, tryptophan and valine.

18. The method according to claim 17, wherein the essential amino acid is selected from the group consisting of leucine, threonine and tryptophan.

19. The method according to claim 14, further comprising a step of measuring in the biological sample to obtain dimethylarginine and a ratio of dimethylarginine/arginine.

20. The method according to claim 14, further comprising a step of measuring in the biological sample to obtain symmetric dimethylarginine and a ratio of symmetric dimethylarginine/arginine.

21. A diagnostic device for diagnosing heart failure comprising:
a detector for detecting a biomarker selected from the group consisting of xanthine, spermidine, propionylcarnitine, butyrylcarnitine, P-cresyl sulfate and a combination thereof.
